(19) United States
(12) Patent Application Publication
(10) Pub. No.: US 2023/0330162 A1
(43) Pub. Date: Oct. 19, 2023

(54) PRODUCT COMPRISING PROBIOTICS AND ISOMALTULOSE AND METHOD OF ITS PRODUCTION

(71) Applicant: Bluestone Pharma GmbH, Baar (CH)

(72) Inventors: Danijela GAVRILOVIC, Leskovac (RS); Milan DMITROVIC, Belgrade (RS); Michael FABER, Sitges (ES); Karsten BRANDT, Sitges (ES)

(21) Appl. No.: 18/134,210

(22) Filed: Apr. 13, 2023

(30) Foreign Application Priority Data
Apr. 13, 2022 (EP) 22 168 236.2

(51) Int. Cl. A61K 35/744; C12N 1/20; A61K 9/20; A61K 9/00; A61K 47/26

(52) U.S. Cl. CPC A61K 35/744; C12N 1/20; A61K 9/2018; A61K 9/2095; A61K 9/0056; A61K 47/26

(57) ABSTRACT

Provided is a product, in particular a compressed product and a powder product, comprising probiotically active microorganisms as active ingredients and isomaltulose as excipient as well as a method for producing such a product.

### FIELD OF THE INVENTION

The invention relates to a product, in particular to powder products and compressed products, which comprise probiotic bacteria as active ingredient and isomaltulose as excipient. Furthermore, the present invention is directed to a method for producing such a product as well as to the use of isomaltulose as excipient in a product comprising probiotic bacteria.

### BACKGROUND OF THE INVENTION

Probiotics are defined as live microorganisms which when administered in adequate amounts confer a health benefit on the host. The beneficial effects of probiotics may be mediated by a direct antagonistic effect against specific groups of undesired organisms, resulting in a decrease of their numbers, by an effect on the metabolism of such groups of organisms or by a general stimulatory effect on the immune system of animal or human hosts.

Probiotic microorganisms have been identified among microorganisms classified as yeasts, fungi and bacteria. For instance, lactic acid bacteria are in general recognized as being useful as probiotics or “probiotically active” organisms, i.e. organisms that may beneficially affect animal or human hosts.

Compressed tablets and powder formulations are the dominant dosage form for self-administration of pharmaceutical compositions. Both products can be produced at a high speed and consequently at low prices and consumers are used to and frequently prefer these dosage forms. Furthermore, lozenges and powders are a particular preferred form especially for children since sucking a lozenge or administering a powder is regarded as more convenient than swallowing a tablet. Lozenges and powders are also particularly useful for the administration of probiotic bacteria conferring beneficial effects to the mouth microflora, like Streptococcus salivarius, since sucking of the lozenge (instead of mere swallowing) and administering a powder which dissolves in the mouth and thus, remains there for a certain time, allows the probiotic bacteria to colonize the oral cavity.

In this context, sugar free lozenges and powder products are becoming increasingly interesting for dietary reasons and for the sake of dental health. Furthermore, the suggested minimal concentration for probiotic bacteria is 10⁶ CFU/g of a product (Shah (2000), J Dairy Sci 83(4):894-907) to provide health benefits, and thus, there is a constant need to provide healthy and consumer friendly products which contain a therapeutically effective amount of viable probiotically active organisms.

### SUMMARY OF THE INVENTION

The present invention relates to the surprising finding that probiotically active organisms can be formulated with isomaltulose as excipient, in particular as bulking agent, wherein this composition can be formulated into tablets, in particular lozenges by compression in a tablet press, and into powder products usually packed in sachets while retaining sufficient amounts of probiotically active organisms.

The inventors have surprisingly shown that a lozenge comprising probiotic bacteria and isomaltulose instead of isomalt has superior properties that could not have been expected. In particular, as can be derived from the Examples, the CFUs of S. salivarius K12 are much higher in the isomaltulose lozenges than in the isomalt lozenges. Although the same concentration of freeze-dried S. salivarius K12 has been used as raw material, more viable bacteria are present in the isomaltulose lozenges in comparison to the corresponding lozenges with isomalt. Furthermore, the disintegration time of the isomaltulose lozenges is higher than the disintegration time of the isomalt lozenges and thus, the isomaltulose lozenges are sucked longer before they disintegrate than the isomalt lozenges, which has i.a. the advantage that there is more time for the oral probiotic to colonize the oral cavity. The friability of both lozenges is comparable and thus, both are stable. Furthermore, the average water activity of the isomaltulose lozenges is lower than the water activity of the isomalt lozenge and thus, isomaltulose lozenges have a longer shelf life.

As regards the sachets, i.e. the powder product, it has been shown that the one comprising isomaltulose as excipient is as storage stable as the one comprising maltodextrin.

In particular, as can be derived from Table 3, the isomaltulose lozenges comprise even after 12 months storage a CFU/g amount of probiotics which is higher than 10⁶ CFU/g which is regarded as the minimal concentration to provide health benefits (Shah (2000, supra)). Similarly, as can be derived from Table 7, the isomaltulose sachets comprise after 6 months storage a CFU/g amount of probiotics which is higher than 10⁶ CFU/g and it is prudent to expect that the CFU/g amount does not drop below 10⁶ CFU/g even after 24 months storage (as it has been shown for the corresponding maltodextrin sachets; see Table 8) since the decrease of CFU/g within the first 6 months of the isomaltulose and the maltodextrin sachets is similar.

Furthermore, the isomaltulose lozenges have a longer disintegration time than the isomalt lozenges, i.e. of more than 10 minutes in comparison to about 5 minutes, allowing an efficient colonialization of mouth probiotics in the oral cavity; see Tables 3, 4, 5 and 6. The lozenges and the sachets also show a low water activity so that they can be regarded as stable and as having a long shelf life. The low friability which has been measured for the lozenges show that those have a high quality; see Tables 3 to 8.

Usually, frequently used excipients like fructose and maltodextrin are substituted with sugar alcohols, like isomalt to provide sugar free lozenges and powder products. However, also the consumption of isomalt has disadvantages, since for example an excessive consumption may have a laxative effect (“604. Isomalt (WHO Food Additives Series 20)”; Retrieved Sep. 28, 2017; and Bachmann et al., (1984), Investigations of the metabolic effects of acute doses of Palatinit®, Akt. Ernahr. 9 (1984), 65-70).

One advantage of isomaltulose is that—in comparison to sucrose and most other carbohydrates—it is “kind to teeth” since it is not a significant substrate for oral bacteria and thus, acid production from isomaltulose in the mouth is too slow to promote tooth decay. Another advantage of the use of isomaltulose as excipient for the production of compressed products is that the tablets can be pressed directly without the use of a binder and without controlled granulation as shown in the Examples.

Accordingly, the present invention provides a product, preferably a compressed product and a powder product which is kind to teeth and consumer friendly. In particular, the present invention provides a product, preferably a compressed product and a powder product which comprises at least probiotic bacteria as active ingredient and isomaltulose as excipient. In a preferred embodiment, the compressed product is a tablet and more preferably a lozenge or a chewable tablet. The product preferably comprises lactic acid bacteria which are in general recognized as being useful as probiotics or “probiotically active” organisms, i.e. organisms that may beneficially affect animal or human hosts.

The product of the present invention comprises in a preferred embodiment bacteria of the genus Streptococcus, Lactobacillus, Lactococcus, Bifidobacterium and/or Enterococcus, or a mixture of any one thereof. Preferably, the product of the present invention comprises bacteria selected from the group consisting of, but not limited thereto: Streptococcus salivarius, preferably strain K12 or strain ENT-K12, M18, 24SMB, Rosell®-83, or HA-188, but more preferably strain K12, ENT-K12, or M18 and most preferably strain K12 and ENT-K12, respectively, Lactobacillus rhamnosus, preferably strain LGG, Lactobacillus casei, preferably strain 431, Lactobacillus paracasei, preferably strain LP-33 (also designated as GMNL-32 or GM-080) or strain GMNL-133, Lactobacillus fermentum, preferably strain LC40, Lactobacillus crispatus, preferably strain M247, Bifidobacterium animalis subsp. lactis, preferably strain BB-12, Lactobacillus plantarum, preferably strain 299v, Lactococcus lactis, Enterococcus faecalis, Lactobacillus reuteri protectis, and Streptococcus dentisani, or a mixture of any one thereof, preferably of two of the mentioned strains.

As it is commonly known in the art and shown in several clinical studies, probiotic organisms have a health benefitting effect and can even be used for the treatment of various diseases; see for example Wescombe et al., Future Microbiol. (2012) 7(12), 1355-1371; Zupancic et al., Probiotics Antimicrob Proteins (2017) 9(2), 102-110; Wilcox et al., Clin Microbiol Infect (2019) 25(6), 673-680; Marom et al., Medicine (Baltimore) (2016) 95(6), e2695; Clark, Curr Opin Immunol (2020) 66, 42-49; Bertuccioli et al., Nutrafoods (2019) 2, 80-88. Accordingly, the present invention further relates to the product for use in the treatment of otitis, preferably otitis media; upper respiratory tract infections, preferably tonsillitis or pharyngitis; lower respiratory tract infections, preferably bronchitis or pneumonia; diseases and inflammations of the oral cavity, preferably caries, oral mucositis, periodontitis, candidiasis, and/or oral lichen planus; halitosis; skin disorders, preferably acne and/or dermatitis; gastro-intestinal problems; allergies or immune diseases preferably allergic rhinitis, or mastitis in a subject.

Furthermore, the product can be used for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form in a subject.

The present invention further relates to a method of producing a product comprising probiotic bacteria and isomaltulose, wherein the method comprises at least mixing the probiotic bacteria with isomaltulose, optionally with further excipients and/or active ingredients. The method for producing a compressed product of the present invention further comprises the compression of the mixture. To guarantees a low water activity of the product, the excipients are dried before tablet pressing. Furthermore, during tablet pressing, the generation of excessive heat is avoided and pressing is performed with a pressure which allows the compaction of the mixture but without being lethal for the probiotic organism.

Further embodiments of the present invention will be apparent from the description, the Figures and Examples that follow.

### BRIEF DESCRIPTION OF THE DRAWINGS

FIG. 1: Exemplarily manufacturing flowsheet for compressed products comprising probiotics, in particular S. salivarius K12 and isomaltulose by dry compression.

FIG. 2: Exemplarily manufacturing flowsheet for compressed products comprising probiotics, in particular S. salivarius K12 and isomaltulose including a wet granulation step.

### DETAILED DESCRIPTION OF THE INVENTION

The present invention relates to a product/composition which comprises at least probiotic microorganism as active ingredient and isomaltulose as excipient. In particular, the inventors found a generally useful probiotic composition in the form of a product, preferably in the form of a compressed product or in the form of a powder product, like a sachet comprising viable, probiotically active organisms, and a method for its production. As regards the compressed product, the inventors found a production method by which the probiotic tablet dosage form can be produced by dry compression in a tablet press comprising probiotically active organisms and isomaltulose as a bulking agent. Furthermore, the experiments performed in accordance with the present invention showed that said probiotic tablet dosage form can also be produced by tablet pressing including a wet granulation step. Thus, the present invention also relates to the use of isomaltulose as excipient in the product, and in particular in the compressed product and the powder product of the present invention.

When features/embodiments/characteristics are described in connection with “the product” or “the product of the present invention”, those generally relate to a corresponding composition and also apply to the compressed product as well as to the powder product if not indicated otherwise. Thus, all features described in regard to “the product” or “the product of the present invention” are features which are attributed to the compressed product and to the powder product if not indicated otherwise.

In one embodiment, the product of the present invention is a compressed product and preferably a tablet. Tablets are solid dosage forms usually obtained by single or multiple compression of powders or granules. They can be either coated or uncoated. Tablets are normally right circular solid cylinders, the end surfaces of which are flat or convex and the edges of which may be beveled. They may have lines or break-marks (scoring), symbols, or other markings. Tablets are single-dose preparations intended for oral administration. Some are intended to be swallowed whole, some after being chewed and some after being crushed, some are intended to be dissolved or dispersed in water before being taken and some are intended to be retained in the mouth where the active ingredient(s) is/are liberated.

The different categories of tablets include: uncoated tablets; coated tablets (including film-coated and sugar-coated tablets); soluble tablets; dispersible tablets; effervescent tablets; chewable tablets; tablets for use in the mouth (including sublingual and buccal tablets); and modified-release tablets (including delayed-release tablets (gastro-resistant/enteric-coated tablets) and sustained-release tablets (extended-/prolonged-release tablets)).

Tablets for use in the mouth like chewable tablets and lozenges, most preferably lozenges are a favored delivery form since especially children associate a lozenge with a candy and since sucking a tablet is generally more convenient than swallowing a whole tablet leading to a high acceptance of such a product. Thus, in a preferred embodiment, the compressed product of the present invention is a tablet for use in the mouth, preferably a chewable tablet or a lozenge, most preferably a lozenge.

Chewable tablets are usually uncoated and are intended to be chewed before being swallowed. Tablets for use in the mouth are also usually uncoated and they are usually formulated to effect a slow release and local action of the active ingredient(s) (for example, compressed lozenges) or the release and absorption of the active ingredient(s) under the tongue (sublingual tablets) or in other parts of the mouth (buccal) for systemic action.

In another embodiment, the product of the present invention is a powder product and preferably an oral powder, i.e. a powder for oral administration. Oral powders are preparations consisting of solid, loose, dry particles of varying degrees of fine particle size. They contain one or more active substances, with or without excipients and, if necessary, approved coloring matter and flavoring. They are generally administered in or with water or another suitable liquid, or they may also be swallowed directly.

Isomaltulose is a disaccharide carbohydrate composed of glucose and fructose and it tastes similar to sucrose (table sugar) with half the sweetness. The glucose and fructose are linked by an alpha-1,6-glycosidic bond (chemical name: 6-0-α-D-glucopyranosyl-D-fructose). Isomaltulose, also known by the trade name Palatinose, is manufactured by enzymatic rearrangement (isomerization) of sucrose from beet sugar. In European Patent 0 028 905 B1, which content is herein incorporated by reference, the use of isomaltulose as diluent in tablets in generally described as well as methods of producing such tablets.

One advantage of isomaltulose is that—in comparison to sucrose and most other carbohydrates—it is not a significant substrate for oral bacteria. Consequently, acid production from isomaltulose in the mouth is too slow to promote tooth decay and thus isomaltulose is “kind to teeth”. Fermentation of carbohydrates by bacteria in the mouth (especially on the teeth) is responsible for the formation of dental plaque and oral acids. The acid initiates tooth demineralization and tooth decay (dental caries). Isomaltulose largely resists fermentation by oral bacteria and is the first carbohydrate of its kind with negligible acid production on teeth, as shown by pH telemetry. The evidence is strong and provides the basis for “kind to teeth” claims approved by both the Food and Drug Administration in the USA and European authorities following a positive opinion from the European Food Safety Authority. In addition, the product of the present invention has been considered “toothfriendly” as determined by standardized in vivo pH-telemetry tests conducted by test facilities accredited by Aktion Zahnfreundlich (Switzerland). A product is considered “toothfriendly” if it lacks a significant cariogenic and erosive potential in healthy people under usual conditions of use.

This is of particular relevance for tablets and powders for use in the mouth like the compressed product and the powder formulation of the present invention.

Another advantage of the use of isomaltulose as a diluent for the production of compressed products is that the tablets can be pressed directly without the use of a binder and without controlled granulation. The method of production of the product of the present invention will be explained in detail further below.

A further favorable feature of the compressed product of the present invention, which seems to be attributable to the presence of isomaltulose, is its long disintegration time. In particular, the compressed product of the present invention has a longer disintegration time than corresponding tablets with isomalt as excipient as shown in the Examples. Due to this long disintegration time, the compressed product remains longer in the oral cavity during sucking. This is particular useful when probiotic bacteria are comprised in the compressed product which are for use in the treatment of upper and lower respiratory tract infections, diseases and inflammations of the oral cavity, and halitosis or which are used for supporting healthy mouth microflora, healthy upper and lower respiratory tract microflora since the longer sucking time allows the probiotic bacteria to efficiently colonize the oral cavity and the respiratory tract. In this context, the powder formulation is also very suitable since it dissolves in the mouth and remains in the oral cavity during dissolution and thus, it also remains sufficiently long in the oral cavity to allow colonialization.

Since isomaltulose is “kind to teeth”, the long sucking time of the compressed product and the distribution of the powder product in the oral cavity, respectively has no negative effect on the health of teeth and even when the product comprises probiotic bacteria which rather act in the gastro-intestinal tract, the compressed product or the sachet is preferably used since for example lozenges rather reminds on a candy than on a medicament or health stimulating agent. Furthermore, for a fast majority of people, sucking a tablet or administering a powder is more convenient than swallowing a tablet.

Thus, in one embodiment, the compressed product of the present invention has a disintegration time of more than 5 minutes, preferably between 10 and 30 minutes, more preferably between 10 and 15 minutes and most preferably between 10 and 11 minutes. In comparison, a corresponding tablet comprising isomalt as excipient has a disintegration time of about 5 minutes; see the Examples.

Furthermore, the amount of viable probiotically active microorganisms, in particular of Streptococcus salivarius, was higher in tablets comprising isomaltulose as excipient than in corresponding tablets comprising isomalt although the same amount of bacteria has been used for producing the compressed products; see Example 1. Accordingly, the formulation of the present invention, i.e. the use of isomaltulose in the product of the present invention, has a beneficial effect on the survival of the probiotically active microorganisms.

As can be derived from Example 1 and Table 3, during manufacturing of the tablets comprising isomaltulose as excipient, the viability of the tablets decreased only from 1.30E+10 CFU/g and 1.04E+10 CFU/g, respectively prior to compression to 1.22E+10 CFU/g and 9.60E+09 CFU/g directly after compression, i.e., in the freshly prepared tablet composition. This corresponds to a decrease in viability of the cells of only about 7% in average during tablet pressing. Thus, the use isomaltulose as excipient in the compressed product of the present invention results in that the viability of the cells during compression in a tablet press does not decrease by more than 10%, preferably by no more than 8%, and in particular by no more than 7%. The decrease in viability of the cells is calculated as: ([the total number of viable cells present in 1 g of the mixture prior to compression]−[the total number of cells present in 1 g of the freshly prepared tablet composition]) divided by [the total number of viable cells present in 1 g of the mixture prior to compression], multiplied by [100%].

Furthermore, the compressed product of the present invention has a hardness (resistance to crushing) of about 70 to 85 N and 6 to 10 Kp, respectively, which is comparable to the hardness of corresponding isomalt tablets. Tablet hardness is the force (load) required to break a tablet. The compressed product of the present invention further has a friability of about 1.2 to 1.5%, which is comparable to the friability of the corresponding isomalt tablets. Friability describes the tendency of a solid substance to break into smaller pieces under duress or contact.

The water activity of a product is important for its shelf-life. In particular, a low water activity ensures a high survival rate of the probiotic bacteria which is important for the effectiveness of the product of the present invention. The water activity of the compressed product of the present invention directly after compression is between about 0.1 and 0.2, preferably between 0.11 and 0.15 and in average of about 0.14 and slightly increases over time. The water activity of the corresponding isomalt product is higher as can be derived from the Examples. The water activity of the powder product of the present invention is between about 0.12 and 0.13 after production and thus both products have a high shelf-life. Thus, the water activity of the product of the present invention is between 0.11 and 0.15 after production.

The International Scientific Association for Probiotics and Prebiotics defines “probiotics” as “live microorganisms that, when administered in adequate amounts, confer a health benefit on the host”. These microorganisms, which consist mainly of bacteria but also include yeasts, are naturally present in fermented foods, may be added to other food products, and are available as dietary supplements, like the product of the present invention. In principle any probiotic strain can be formulated in the product of the present invention. Health benefits have mainly been demonstrated for specific probiotic strains of the following genera: Aerococcus, Bacillus, Bacteroides, Bifidobacterium, Clostridium, Enterococcus, Fusobactehum, Lactobacillus, Lactococcus, Leuconostoc, Melissococcus, Micrococcus, Pediococcus, Peptostrepococcus, Propionibacterium, Staphylococcus, Streptococcus and Weissella. Thus, the product of the present invention, in particular the compressed product and the powder product, preferably comprises species of any one of those genera or mixtures thereof.

Common microorganisms which may be formulated in the product of the present invention include but are not limited to the following: Lactic acid bacteria: Genus Lactobacilli spp.; Species: Lactobacillus acidophilus, L. alimentarius, L. amylovorus, L. brevis, L. bulgaricus, L. casei, L. cellobiosus, L. crispatus, L. curvatus, L. delbrueckii spp. bulgaris, L. delbrueckii spp. lactis, L. farciminus, L. fermentum, L. gallinarum, L. helveticus, L. johnsonii, L. lactis, L. paracasei, L. plantarum, L. reuteri, L. rhamnosus, L. sake, L. salivarius; Genus: Streptococcus spp. Species: Streptococcus salivaris spp., S. salivarius (for example K12 or ENT-K12), S. dentisani, S. faecalis, S. faecium; Genus: Lactococcus ssp., Species: L. lactis cremoris, L. lactis; Genus: Leuconostoc, Species: Lc. mesenteroides; and Genus: Pediococcus spp., Species: P. pentosaceus, P. acidilactici.

Bifidobacteria: Genus: Bifidobacterium spp., Species: B. adolescentis, B. animalis, B. bifidum, B. breve, B. essensis, B. infantis, B. laterosporum, B. thermophilum, B. longum.

Propionibacteria: Genus: Propionibacterium spp., Species: P. acidipropionici, P. freudenreichii, P. jensenii, P. thoenii.

Enterobacteria: Genus: Enterococcus spp., Species: E. fecalis, E. faecium. E. durans.

Sporulated bacteria: Genus: Bacillus spp., Species: B. alcolophilus, B. cereus, B. clausii, B. coagulans, B. subtilis.

Other bacteria: Genus: Escherichia coli, Species: E. coli; Genus: Sporolactobacillus spp.

Species: S. inulinus.

Yeasts: Genus: Saccharomyces spp., Species: S. cerevisae (boulardii); that isolated from litchi fruit in Indonesia have also been accepted and used as probiotics.

In a preferred embodiment, the product of the present invention comprises lactic acid bacteria or bifidobacteria, preferably those mentioned above. As used herein the term “lactic acid bacteria” refers to gram-positive, microaerophilic or anaerobic bacteria which ferment sugars with the production of acids including lactic acid as the predominantly produced acid, acetic acid, formic acid and propionic acid. Lactic acid bacteria are particularly preferred.

In a particular preferred embodiment, the probiotic bacteria are selected form the group consisting of: Streptococcus salivarius, preferably strain K12 (DSM 13084; BAA-1024), ENT-K12 (DSM 34540), 24SMB (DSM 23307), M18 (ATCC BAA-2593; DSM 14685), Rosell®-83, or HA-188, but more preferably strain K12 or M18, and most preferably strain K12 and ENT-K12, respectively, Lactobacillus rhamnosus, preferably strain LGG (ATCC 53103; U.S. Pat. No. 4,839,28), Lactobacillus casei, preferably strain 431 (ATCC 55544), Lactobacillus paracasei, preferably strain LP-33 (also designated as GMNL-32 or GM-080; CCTCC M 204012, U.S. Pat. No. 6,994,848 B2) or strain GMNL-133 (CCTCC M 2011331, EP 2 581 461 B1), Lactobacillus fermentum, preferably strain LC40 (CECT5716, U.S. Pat. No. 7,468,270 B2), Lactobacillus crispatus, preferably strain M247 (LMG P-23257, EP 1 930 018 A1), Bifidobacterium animalis subsp. Lactis, preferably strain BB-12 (DSM 15954, EP 2 990 045 B1), Lactobacillus plantarum, preferably strain 299v (DSM 9843), Lactococcus lactis (Zamfir et al. 2016 Int J Food Sci Technol, 51, 2164-2170), Enterococcus faecalis (DSM 16440), Lactobacillus reuteri protectis, (DSM 17938); Streptococcus dentisani (CECT7746), and/or any combinations thereof.

An accession number starting with “DSM” indicates that the strain is deposited with the Deutsche Sammlung von Mikroorganismen Und Zellkulturen GmbH (DSMZ), Mascheroder Weg lb, D-38124 Braunschweig, GERMANY; a number starting with “ATCC” indicates that the strain is deposited with the American Type Culture Collection (ATCC), P.O. Box 1549, Manassas, VA 20108, USA; a number starting with “CCTCC” indicates that the strain is deposited with the China Center for Type Culture Collection (CCTCC), College of Life Sciences, Wuhan University Wuhan 430072, China; a number starting with “CETC” indicates that the strain is deposited with the Colección Espanola de Cultivos Tipo (CECT), Edificio 3 CUE, Parc Cientific Universitat de Valencia, Catedrático Augustín Escardino 9, 46980 Paterna (Valencia); a number starting with “LMG” indicates that the strain is deposited with the BCCM/LMG Bacteria Collection, Laboratorium voor Microbiologie, Ghent University, K. L. Ledeganckstraat 35, B-9000 Ghent, Belgium.

In one preferred embodiment, the strain is S. salivarius K12, which is publicly available at the American Type Culture Collection (ATCC), P.O. Box 1549, Manassas, VA 20108, USA under Accession No. BAA-1024.

In one preferred embodiment, the strain is S. salivarius ENT-K12, which is genetically identical to S. salivarius K12 and which has been deposited at the Leibniz-Institut DSMZ—Deutsche Sammlung von Mikroorganismen und Zellkulturen GmbH, Inhoffenstraße 7B, 38124 Braunschweig, Germany, and assigned Accession Number DSM 34540 (Date of deposit: Feb. 22, 2023). The depositor is the Probionet GmbH, Schutzenstrasse 380, 9100 Herisau, Switzerland.

The invention is not, however, limited to these above-mentioned particular microorganisms. The person skilled in the art would recognize those microorganisms, which may be useful in the product according to the invention. A very important embodiment of the present invention is to combine two or more of the above mentioned probiotically active organisms, such as e.g. a preparation comprising a probiotically active Lactobacillus species and a probiotically active Streptococcus species.

The product of the present invention preferably comprises the following probiotically active organisms:
Streptococcus salivarius, preferably K12, ENT-K12 or M18
Lactobacillus rhamnosus, preferably strain LGG
Lactobacillus casei, preferably strain 431
Lactobacillus paracasei, preferably strain LP-33 and/or GMNL-133
Lactobacillus fermentum, preferably strain LC40
Lactobacillus crispatus, preferably strain M247
Lactobacillus plantarum, preferably strain 299v
Bifidobacterium animalis subsp. lactis, preferably strain BB-12
Lactococcus lactis
Streptococcus salivarius and Lactobacillus rhamnosus, preferably Streptococcus salivarius K12 or ENT-K12, and Lactobacillus rhamnosus strain LGG;
Streptococcus salivarius and Lactobacillus casei, preferably Streptococcus salivarius K12 or ENT-K12, and Lactobacillus casei 431;
Streptococcus salivarius and Lactobacillus fermentum, preferably Streptococcus salivarius K12 or ENT-K12, and Lactobacillus fermentum strain LC40;
Streptococcus salivarius and Lactobacillus crispatus, preferably Streptococcus salivarius K12 or ENT-K12, and Lactobacillus crispatus strain M247;
Streptococcus salivarius and Lactobacillus plantarum, preferably Streptococcus salivarius K12 or ENT-K12, and Lactobacillus plantarum strain 299v;
Streptococcus salivarius and Bifidobacterium animalis subsp. lactis, preferably Streptococcus salivarius K12 or ENT-K12, and Bifidobacterium animalis subsp. lactis strain BB-12;
Streptococcus salivarius and Lactococcus lactis, preferably Streptococcus salivarius K12 or ENT-K12, and Lactococcus lactis;
Streptococcus salivarius and Lactobacillus paracasei, preferably Streptococcus salivarius K12 or ENT-K12, and Lactobacillus paracasei LP-33;
Streptococcus salivarius and Lactobacillus paracasei, preferably Streptococcus salivarius K12 or ENT-K12, and Lactobacillus paracasei GMNL-133.

In one embodiment, any of the above-mentioned combinations which include a Streptococcus salivarius strain include Streptococcus salivarius K12.

In one embodiment, any of the above-mentioned combinations which include a Streptococcus salivarius strain include Streptococcus salivarius ENT-K12.

Of course, combinations any other of the above-mentioned strains and/or of three, four or more of the above-mentioned strains are also possible and encompassed.

Next to the probiotic microorganisms and isomaltulose, the product of the present invention comprises in one embodiment one or more further excipients and/or active ingredients.

The further active ingredients can be compounds that support and/or boost the immune system like vitamins and minerals. Those include, but are not limited to vitamin A, vitamin D3, vitamin B6, vitamin C, folic acid, vitamin B12, iron, zinc, copper, selenium and combinations thereof. In a preferred embodiment, the further active ingredient is vitamin D3 (cholecalciferol), for example in a form of a premix.

A further active ingredient, which is preferably present in the powder product of the present invention are fructooligosaccharides (FOS). FOS are oligosaccharide fructans and have a number of interesting properties, including a low sweetness intensity; they are also calorie free, non-cariogenic and are considered as soluble dietary fibre. Furthermore, FOS have important beneficial physiological effects such as a prebiotic effect, improved mineral absorption and decreased levels of serum cholesterol, triacylglycerols and phospholipids. Furthermore, FOS has GRAS status (Generally recognized as safe is a United States Food and Drug Administration (FDA) designation that a chemical or substance added to food is considered safe by experts).

Excipients are helpful and/or necessary to prepare solid dosage forms. They are widely involved in the flow properties of mixtures, compression properties in tablet preparation, disintegration properties for smooth disintegration, sticking properties in which powder sticks to the surface of tablet compression punches to cause defects on the tablet surface, capping in which the upper portion of tablets is peeled off in a cap shape during compression, lamination in which a tablet is peeled off in a layered fashion, and binding properties for enhancing the hardness of tablets.

Excipients include bulking agents, diluents, carrier, binders, lubricants/anti-caking agents, disintegrators, preservatives, colors or flavors. The excipients are preferably pharmaceutically acceptable and/or acceptable for human consumption.

Acceptable carrier/bulking agents suitable for use in the administration of viable probiotic microorganisms are well known to those skilled in the art; see, for example, Remington's Pharmaceutical Sciences, 18th ed., Gennaro, ed., 1990, Mack Publishing Co., Easton, Pa., incorporated herein by reference. Preferably, the carrier/bulking agent is a pharmaceutically acceptable carrier/bulking agent and/or a carrier/bulking agent acceptable for human consumption. Acceptable carriers/bulking agents suitable for use with probiotically active microorganisms in the compressed product of the present invention are usually solid carriers known in the art and include, but are not limited to magnesium carbonate; magnesium stearate; celluloses; talc; sugars such as fructose, sucrose, mannitol, sorbitol, xylitol, lactose; sugar substitutes such as isomalt; starches; maltodextrin; flours; (fructose-)oligosaccharides and skim milk, and similar edible powders, but are not limited thereto.

Typical diluents, by way of example, are: starches; lactose; mannitol; kaolin; calcium phosphate or sulphate; inorganic salts such as sodium chloride; and powdered sugars and sugar substitutes as mentioned above or celluloses.

Typical binders include starch; gelatin; sugars such as lactose, fructose, and glucose; and the like. Natural and synthetic gums are also convenient, including acacia; alginates; locust bean gum; methylcellulose, e.g., Hydroxypropyl methylcellulose (HMPC); polyvinylpyrrolidine (PVP) tragacanth; PVP K-30; PVP K-25; xanthan gum: and the like. Polyethylene glycol (PEG 4000 or PEG 6000); ethyl cellulose; and waxes can also serve as binders as well as Nu-BIND® and CompactCel®DIS.

Lubricants and anti-caking agents to prevent sticking during formulation and to prevent the formation of lumps include slippery solids such as talc, silica, magnesium and calcium stearate, polyethylene glycol, stearic acid, hydrogenated vegetable oils, rice extract blend, for example Nu-MAG®, CompactCel®LUB, in particular CompactCel® F clear 290.02 LUB, oat fiber blend, for example CompactCel® F 200.28 LUB, potato starch, gum Arabica, CompactCel®FLO, Nu-FLOW®, silicon dioxide, tricalcium phosphate, and rice hulls, for example Nu-FLOW®, or CompactCel®FLO.

Disintegrators are substances which swell when wetted to break up the composition and release the S. salivarius or extract. The disintegrators include starches; clays; celluloses; algins and gums; more particularly corn and potato starches; methylcellulose; agar; bentonite; wood cellulose; cation exchange resins; alginic acid; guar gum; citrus pulp; carboxymethylcellulose; powdered sponge; silica; and sodium lauryl sulfate.

Aromatizing agents are known to the person skilled in the art and can be of any kind which give a good (or at least a different taste) to the compressed product. Those flavors include, but are not limited to strawberry, mint, orange, banana, passionfruit, cocoa, menthol, yuzu, lemon and/or combinations thereof, preferably passionfruit, cocoa and menthol; yuzu and mint; orange and mint.

Powder formulations can be in principle produced comprising only the active ingredient, here the probiotic but preferably, similar as mentioned for the compressed product above, excipients are present and in particular, for example those mentioned above, i.e. carrier, lubricants, anti-caking agents, disintegrators, preservatives, colors and/or flavors. The excipients are preferably pharmaceutically acceptable and/or acceptable for human consumption.

In one embodiment, the compresses product of the present invention further comprises a lubricant/anti-caking agent and optionally a flavor. This relates in particular to a compressed product produced by dry compression. In one embodiment, the compressed product of the present invention further comprises a lubricant/anti-caking agent and a binder and optionally a flavor. This relates in particular to a compressed product produced by compression including a wet granulation step. In one embodiment, the powder product of the present invention does not comprise any further excipient, but optionally further comprises an anti-caking agent or a flavor. In one embodiment, the powder product of the present invention does not comprise any further excipient, but optionally further comprises an anti-caking agent and a flavor.

In order to obtain a sweet taste of the product of the present invention, the compressed products, in particular the tablets, and powder products further comprise Stevia. Stevia is a natural sweetener and sugar substitute derived from the leaves of the plant species Stevia rebaudiana. Thus, in one embodiment, the product of the present invention further comprises a natural sweetener, preferably Stevia.

In recent years, as consumer interest in well-being and natural foods has increased, the use of natural additives in place of synthetic additives in the food industry has gradually increased as well as the use of sugar substitutes. In particular, in pharmaceutical drugs and health functional foods, safety and environmentally friendly factors in production processes are considered important, and thus many products containing natural components are being developed and the market size thereof is also increasing.

Accordingly, in a preferred embodiment, the product of the present invention does not comprise a sugar, but only the sugar substitute isomaltulose and optionally (in particular in case of the powder product) FOS, and further optionally the natural sweetener Stevia. Furthermore, in one embodiment the product, in particular the compressed product of the present invention does comprise as few additives as possible and thus, only further comprises (next to isomaltulose and the probiotic bacteria) a lubricant/anti-caking agent as excipient, but no other excipients like binders or disintegrators or does even comprise no further excipients. In particular, in one embodiment the compressed product of the present invention only further comprises a lubricant/anti-caking agent as excipient, but no other excipients like binders or disintegrators.

In one embodiment, the product, in particular the compressed product of the present invention does comprise as few additives as possible and thus, only further comprises (next to isomaltulose and the probiotic bacteria) a lubricant/anti-caking agent and a binder as excipients, but no other excipients like disintegrators.

In one embodiment, the powder product of the present invention which comprises probiotic bacteria, isomaltulose and optionally FOS, does not comprise any further excipients, but may optionally comprise an anti-caking agent as excipient which becomes especially important for oversea business, especially during air freight.

However, the product of the present invention may comprise an aromatizing agent, preferably a natural flavor and optionally a natural sweetener, like Stevia.

In a preferred embodiment, the lubricant/anti-caking agent which is comprised in the product of the present invention is magnesium stearate, or a natural replacement. Natural lubricants are known in the art and are for example a crude fat-containing bean powder as described in WO 2013/165131 A1, a rice extract blend, for example the product Nu-MAG®, or the product CompactCel® LUB (CompactCel® F clear 290.02 LUB, Biogrund GmbH, Huenstetten, Germany), an oat fiber blend, for example the product CompactCel® LUB (CompactCel® F 200.28 LUB, Biogrund GmbH, Huenstetten, Germany) or further products, like potato starch, or gum Arabica. In another preferred embodiment, the lubricant/anti-caking agent which is comprised in the product of the present invention is silicon dioxide or tricalcium phosphate or a natural replacement. Natural anti-caking agents are also known in the art and are for example powdered cellulose, like JELUCEL®, native potato starch, inulin, rice fibers, or rice hulls, for example Nu-FLOW®, or CompactCel®FLO. In another preferred embodiment, the binder comprised in the product of the present invention is HMPC or a natural replacement. Natural binders are for example CompactCel®DIS, NuBind®, or pregelatinized corn starch.

In a preferred embodiment, the product, in particular the compressed product of the present invention comprises any one of the probiotic bacteria or combinations of probiotic bacteria listed above, preferably S. salivarius K12 and ENT-K12, respectively, or S. salivarius M18 and a Lactobacillus strain, in particular S. salivarius K12 and L. rhamnosus LGG, or S. salivarius K12 and L. casei 431, or S. salivarius ENT-K12 and L. rhamnosus LGG, or S. salivarius ENT-K12 and L. casei 431, as well as isomaltulose, magnesium stearate and a flavor preferably selected from those mentioned above, most preferably strawberry flavor. Alternatively, magnesium stearate is substituted with a natural lubricant/anti-caking agent, preferably with an oat fiber blend, preferably comprising oat fibers, microcrystalline cellulose and sunflower oil refined, for example the product CompactCel® F 200.28 LUB, or with a rice extract blend, preferably comprising rice extract, microcrystalline cellulose, and sunflower oil refined, for example the product CompactCel® F clear 290.02 LUB, or any one of Nu-MAG®, potato starch, gum Arabica, or with rice hulls, like Nu-FLOW®, or CompactCel® FLO, but most preferably with a rice extract blend in the compressed product of the present invention.

Accordingly, in one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, rice hulls, gum arabic and sunflower oil, for example the product Nu-MAG®, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, microcrystalline cellulose and sunflower oil refined, for example the product CompactCel®LUB, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, an oat fiber blend, preferably comprising oat fiber, microcrystalline cellulose and sunflower oil refined, for example the product CompactCel®LUB, and the above-mentioned flavor, preferably strawberry flavor, and optionally, but preferably a natural sweetener, like Stevia.

In another preferred embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, magnesium stearate, HMPC and a flavor preferably selected from those mentioned above, most preferably strawberry flavor, and optionally, but preferably a natural sweetener, like Stevia. Alternatively, magnesium stearate is substituted with a natural lubricant/anti-caking agent, preferably with an oat fiber blend, preferably comprising oat fibers, microcrystalline cellulose and sunflower oil refined, for example the product CompactCel® F 200.28 LUB, or with a rice extract blend, preferably comprising rice extract, microcrystalline cellulose, and sunflower oil refined, for example the product CompactCel® F clear 290.02 LUB, or a rice extract blend, preferably comprising rice extract, rice hulls, gum arabic and sunflower oil, for example the product Nu-MAG®, or potato starch, gum Arabica, or with rice hulls, like Nu-FLOW®, or CompactCel® FLO, but most preferably with a rice extract blend in the compressed product of the present invention.

Accordingly, in one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, rice hulls, gum arabic and sunflower oil, for example the product Nu-MAG®, HMPC, and the above-mentioned flavor.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, microcrystalline cellulose and sunflower oil refined, for example the product CompactCel®LUB, HMPC, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, an oat fiber blend, preferably comprising oat fiber, microcrystalline cellulose and sunflower oil refined, for example the product CompactCel®LUB, HMPC, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, magnesium stearate, a natural binder like a gum fiber blend, preferably NuBind®, and the above-mentioned flavor preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, magnesium stearate, a natural binder like a gum fiber blend, preferably CompactCel®DIS, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, magnesium stearate, a natural binder like, preferably pregelatinized corn starch, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, rice hulls, gum arabic and sunflower oil, for example the product Nu-MAG®, a natural binder like a gum fiber blend, preferably NuBind®, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, microcrystalline cellulose and sunflower oil refined, for example the product CompactCel®LUB, a natural binder like a gum fiber blend, preferably NuBind®, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, an oat fiber blend, preferably comprising oat fiber, microcrystalline cellulose and sunflower oil refined, for example the product CompactCel®LUB, a natural binder like a gum fiber blend, preferably NuBind®, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, rice hulls, gum arabic and sunflower oil, for example the product Nu-MAG®, a natural binder like a gum fiber blend, preferably CompactCel®DIS, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, microcrystalline cellulose and sunflower oil refined, for example the product CompactCel®LUB, a natural binder like a gum fiber blend, preferably CompactCel®DIS, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, an oat fiber blend, preferably comprising oat fiber, microcrystalline cellulose and sunflower oil refined, for example the product CompactCel®LUB, a natural binder like a gum fiber blend, preferably CompactCel®DIS, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, rice hulls, gum arabic and sunflower oil, for example the product Nu-MAG®®, a natural binder, preferably pregelatinized corn starch, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, microcrystalline cellulose and sunflower oil refined, for example the product CompactCel®LUB, a natural binder, preferably pregelatinized corn starch, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, an oat fiber blend, preferably comprising oat fiber, microcrystalline cellulose and sunflower oil refined, for example the product CompactCel®LUB, a natural binder, preferably pregelatinized corn starch, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, rice hulls, gum arabic and sunflower oil, for example the product Nu-MAG®, a natural binder, preferably pregelatinized corn starch, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, a rice extract blend, preferably comprising rice extract, microcrystalline cellulose and sunflower oil refined, for example the product CompactCel®LUB, a natural binder, preferably pregelatinized corn starch, and the above-mentioned flavor, preferably strawberry flavor, and optionally, but preferably a natural sweetener, like Stevia.

In one embodiment, the product, in particular the compressed product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, an oat fiber blend, preferably comprising oat fiber, microcrystalline cellulose and sunflower oil refined, for example the product CompactCel®LUB, a natural binder, preferably pregelatinized corn starch, and the above-mentioned flavor, preferably strawberry flavor, and optionally, but preferably a natural sweetener, like Stevia.

In another preferred embodiment, the product, in particular the powder product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, FOS, silicon dioxide and a flavor preferably selected from those mentioned above, most preferably strawberry flavor. Alternatively, silicon dioxide is substituted with tricalcium phosphate. Alternatively, silicon dioxide and tricalcium phosphate, respectively, is substituted with a natural anti-caking agent, preferably rice hulls, for example the product Nu-FLOW® or CompactCel® FLO in the powder product of the present invention.

Accordingly, in one embodiment, the product, in particular the powder product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, FOS, rice hulls, preferably Nu-FLOW®, and the above-mentioned flavor, preferably strawberry flavor, and optionally a natural sweetener, like Stevia.

In one embodiment, the product, in particular the powder product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively, and L. casei 431, as well as isomaltulose, FOS, rice hulls, preferably CompactCel® FLO, and the above-mentioned flavor, preferably strawberry flavor, and optionally a natural sweetener, like Stevia.

In another preferred embodiment, the product, in particular the powder product of the present invention comprises any one of the probiotic bacteria or combinations of probiotic bacteria listed above, preferably S. salivarius K12 and S. salivarius ENT-K12, respectively, or S. salivarius K12 and S. salivarius ENT-K12, respectively, and a Lactobacillus strain, in particular S. salivarius K12 and S. salivarius ENT-K12, respectively, and L. rhamnosus LGG, or S. salivarius K12 and S. salivarius ENT-K12, respectively. and L. casei 431, as well as isomaltulose, FOS, and a flavor preferably selected from those mentioned above, most preferably strawberry flavor, preferably strawberry flavor, and optionally a natural sweetener, like Stevia.

Nu-MAG®, Nu-FLOW®, NuBind®, CompactCel®LUB, CompactCel®FLO, and CompactCel®DIS are natural excipients, i.e., natural lubricants, anti-caking agents, and binders, respectively. They are composed of natural organic ingredients and do not comprise for example silica or magnesium stearate and are thus suitable for “clean label” products. For example, Nu-MAG® is a rice extract blend and comprises rice extract, rice hulls, gum arabic and sunflower oil. Nu-FLOW® is a rice concentrate (a concentrate of silica from rice) and comprises rice hulls. NuBind® is a gum fiber blend and comprises guar gum, gum arabic, agave fiber, rice hulls, and agave syrup. CompactCel®LUB is available in two variants, i.e., either comprising rice extract, microcrystalline cellulose and sunflower oil refined, or oat fiber, microcrystalline cellulose and sunflower oil refined.

Optionally, one or more further active ingredients can be used, which are for example those vitamins and minerals mentioned above. In a preferred embodiment, vitamin D3 (cholecalciferol) is used as further active ingredient. Vitamin D3 can be formulated into the product in form of a vitamin D3 premix, which comprises 2.5 μg/mg vitamin D3. In one embodiment, the product of the present invention comprises 4 to 10 μg vitamin D3, i.e. cholecalciferol. In particular, the compressed product preferably comprises 5 to 10 μg vitamin D3 and the powder product 4 to 6 μg vitamin D3.

The contents of the excipient(s) and active ingredient(s) may vary and a person skilled in the art knows how to properly mix the ingredients in order to produce a product which has the desired properties, i.e. the properties of the compressed product as described above regarding CFU amount, water activity, disintegration time, average mass, resistance to crushing, and/or friability, and of the powder product regarding CFU amount and water activity.

Products of the present invention weight at least enough to formulate 5 mg of the probiotically active organisms, preferably to formulate at least 30 mg of the probiotic microorganism and only weight so much that it is still convenient to administer, i.e. to suck the compressed product and to administer the powder product, respectively. The weight of the product can be from about 100 mg to about 2000 mg, wherein the weight of the compressed product ranges preferably from about 500 mg to about 1000 mg, and the weight of the powder product preferably from 500 mg to 1500 mg. Preferably, the compressed product of the invention weighs about 800 mg±10 mg and the powder product about 1500 mg.

The isomaltulose comprises at least 10% and up to 97% of the product of the present invention. Preferably, the amount of isomaltulose is between 50% and 97%, more preferably between 58% and 96%

In one embodiment, the compressed product of the present invention comprises at least 10% and up to 97% isomaltulose. Preferably, the amount of isomaltulose is between 80% and 97%, more preferably between 82% and 92%, more preferably 82.8%, 83.2%, 85.3%, 85.7%, 90.2%, 90.6%, 91.4%, 91.5%, or 91.9% of the compressed product. Assuming that the compressed product of the present invention weights 810 mg, the compressed product comprises between 81 mg and 786 mg isomaltulose, preferably between 648 mg and 786 mg, more preferably between 664 mg and 745 mg, more preferably about 670 mg, 674 mg, 691 mg, 694 mg, 731 mg, 734 mg, 740 mg, 741 mg or 744 mg isomaltulose.

In another embodiment, the isomaltulose comprises at least 10% and up to 97% of the powder product of the present invention. Preferably the amount of isomaltulose is between 50% and 97%, more preferably between 58% and 96%. In particular, the amount of isomaltulose is between 58% and 64%, and most preferably 58%, 60%, 62%, 63% or 64% (in case FOS is additionally present in the powder product), or between 92% and 96% and most preferably 90%, 92%, 94%, 95%, or 96% (in case no FOS is present in the powder product). Assuming that the powder product of the present invention weights 1500 mg, the product comprises between 150 mg and 1455 mg isomaltulose, preferably between 750 mg and 1455 mg, more preferably between 870 mg and 1450 mg. In particular, the amount of isomaltulose in a 1500 mg product is between 870 mg and 970 mg, most preferably 875 mg, 895 mg, 935 mg, 945 mg, 955 mg or 965 mg (in case FOS is additionally present in the powder product), or between 1350 mg and 1450 mg, most preferably 1355 mg, 1375 mg, 1415 mg, 1425 mg, 1435 mg, 1445 mg (in case no FOS is present in the powder product).

The probiotic microorganisms comprise at least 0.3% and up to 20% of the product of the present invention. The product of the present invention preferably comprises between 5 mg to 160 mg probiotic bacteria, more preferably between 30 mg and 120 mg, most preferably 40 mg, 50 mg, 60 mg, 100 mg or 120 mg.

As regards the compressed product, the probiotic microorganisms comprise at least 0.6% and up to 20% of the compressed product of the present invention. Preferably, the content of the probiotic microorganisms is between 3% and 15%, more preferably about 6.2%, 7.4%, 12.3% or 14.8%. The compressed product comprises between 5 mg to 160 mg probiotic bacteria, preferably between 30 mg and 120 mg, more preferably 40 mg, 50 mg, 60 mg, 100 mg or 120 mg. As regards the powder product, the probiotic microorganisms comprise at least 0.3% to 11% of the powder product. Preferably, the content of the probiotic microorganisms is between 2% and 8%, more preferably about 3% (2.7 or 3.3%), 4%, 7% (6.6%), or 8%. The powder product comprises between 5 mg to 160 mg probiotic bacteria, preferably between 30 mg and 120 mg, more preferably 40 mg, 50 mg, 60 mg, 100 mg or 120 mg.

For example, the product of the present invention, in particular the compressed product and the powder product of the present invention, comprises 100 mg or 120 mg of Lactobacillus paracasei in total, either 100 mg or 120 mg of each of the strains LP-33 or GMNL-133 alone or 100 mg or 120 mg of both strains in combination; or 50 mg of Streptococcus salivarius K12 and S. salivarius ENT-K12, respectively; or 40 mg of Streptococcus salivarius M18; or 30 mg of Streptococcus salivarius K12 and S. salivarius ENT-K12, respectively, and 30 mg of Lactobacillus rhamnosus LGG; or 30 mg of Streptococcus salivarius K12 and S. salivarius ENT-K12, respectively, and 30 mg of Lactobacillus casei 431; or 30 mg of Streptococcus salivarius K12 and S. salivarius ENT-K12, respectively, and 30 mg of Bifidobacterium animalis subsp. lactis BB-12; or 20 mg of Streptococcus salivarius K12 and S. salivarius ENT-K12, respectively, 20 mg of Lactobacillus rhamnosus LGG and 20 mg of Bifidobacterium animalis subsp. lactis BB-12; or 20 mg of Streptococcus salivarius K12 and S. salivarius ENT-K12, respectively, 20 mg of Lactobacillus casei and 20 mg of Bifidobacterium animalis subsp. lactis BB-12; or 50 mg of Streptococcus salivarius K12 and S. salivarius ENT-K12, respectively, and 50 mg of Lactobacillus paracasei LP-33; or 50 mg of Streptococcus salivarius K12 and S. salivarius ENT-K12, respectively, and 50 mg of Lactobacillus paracasei GMNL-133.

It will be appreciated that useful probiotically active organisms can be of a genetically modified strain of one of the above organisms. The term “genetically modified” as used herein indicates any modification of DNA sequences coding for genes and modifications of sequences that regulate the expression of genes. Accordingly, genetic modification can be based on construction or selection of mutants of microorganism or it can be based on recombinant DNA technology.

As used herein the term “mutant” is comprised by the conventional meaning of that term, i.e. it refers to strains obtained by subjecting a microbial strain to any conventionally used mutagenization treatment including treatment with a chemical mutagen such as ethanemethane sulphonate (EMS) or N-methyl-N′-nitro-N-nitroguanidine (NTG), UV light or to spontaneously occurring mutants which are selected on the basis of a desired characteristic such as e.g. antibiotic and/or gastric acid-resistance. It is also possible to select useful genetically modified organisms according to the invention by random mutagenesis or by selection of spontaneously occurring mutants, i.e. without the use of recombinant DNA technology. Mutants of the above-mentioned organisms also can be provided by recombinant DNA technology including site-directed mutagenesis, PCR techniques and other in vitro or in vivo modifications and insertion of DNA sequences.

The product of the present invention preferably comprises 10⁵, 10⁶, 10⁹ or even 10¹¹ CFU per g. In a preferred embodiment, the product of the present invention comprises between 5×10⁹ and 10×10⁹ CFUs of the probiotic microorganisms. This will be obtained by using about 40 mg to 120 mg of the probiotic strains when producing the product of the present invention.

As regards the further excipients and active ingredient, the compressed product of the present invention comprises in a preferred embodiment 1% (about 8 mg) magnesium stearate and its natural substitute as defined above, for example a rice extract blend, preferably CompactCel®LUB, respectively, 1% (about 8 mg) aromatizing agent, preferably strawberry flavor, and 0.4% of a vitamin and/or mineral, preferably 5-10 μg vitamin D3 (cholecalciferol), which can be provided for example in a form of a premix (0.4%) which comprises 2.5 μg/mg vitamin D3. Said compressed product is preferably produced by dry compression.

As regards the further excipients and active ingredient, the compressed product of the present invention comprises in another preferred embodiment 1% (about 8 mg) magnesium stearate and its natural substitute as defined above, for example a rice extract blend, preferably CompactCel®LUB, respectively, 1% (about 8 mg) aromatizing agent, preferably strawberry flavor, 0.1% (about 0.74 mg) HMPC, and 0.4% of a vitamin and/or mineral, preferably 5-10 μg vitamin D3 (cholecalciferol), which can be provided for example in a form of a premix (0.4%) which comprises 2.5 μg/mg vitamin D3. Said compressed product is preferably produced by compression including a wet granulation step.

As regards the further excipients and active ingredient, the compressed product of the present invention comprises in another preferred embodiment 1% (about 8 mg) magnesium stearate and preferably its natural substitute as defined above, for example a rice extract blend, preferably CompactCel®LUB, respectively, 1% (about 8 mg) aromatizing agent, preferably strawberry flavor, 0.9% (about 7.42 mg) pregelatinized corn starch, 0.04% (about 0.32 mg) of a natural sweetener, preferably Stevia, and 0.25% of a vitamin and/or mineral, preferably 5-10 μg vitamin D3 (cholecalciferol), which can be provided for example in a form of a premix (0.4%) which comprises 2.5 μg/mg vitamin D3. Said compressed product is preferably produced by compression including a wet granulation step.

An exemplarily compressed product comprises the ingredients as shown in Tables 2A, 2B, and 2C.

TABLE 2A
Ingredients of an exemplarity isomaltulose/probiotics tablet.

| | | Amount | Percentage |
|---|---|---|---|
| | Function | (mg) | (%) |
| Streptococcus salivarius | Active ingredient/ | 50.0 | 6.17 |
| K12/ENT-K12 | probiotic | | |
| | microorganism | | |
| Vitamin D3 premix | Active ingredient | 3.2 | 0.40 |
| Isomaltulose | Bulking agent | 740.8 | 91.45 |
| Magnesium stearate (vegetable) | Anticaking agent, | 8.0 | 0.99 |
| | lubricant | | |
| Strawberry flavor | Flavor | 8.0 | 0.99 |
| Total | | 810 | 100 |

TABLE 2B
Ingredients of an exemplarity isomaltulose/probiotics tablet.

| | | Amount | Percentage |
|---|---|---|---|
| | Function | (mg) | (%) |
| Streptococcus salivarius | Active ingredient/ | 50.0 | 6.17 |
| K12/ENT-K12 | probiotic | | |
| | microorganism | | |
| Vitamin D3 premix | Active ingredient | 3.2 | 0.40 |
| Isomaltulose | Bulking agent | 740.06 | 91.36 |
| Hydroxypropylmethlycellulose | Binder | 0.74 | 0.09 |
| Magnesium stearate (vegetable) | Anticaking agent, | 8.0 | 0.99 |
| | lubricant | | |
| Strawberry flavor | Flavor | 8.0 | 0.99 |
| Total | | 810 | 100 |

TABLE 2C
Ingredients of an exemplarity isomaltulose/probiotics tablet.

| | | Amount | Percentage |
|---|---|---|---|
| | Function | (mg) | (%) |
| Streptococcus salivarius | Active ingredient/ | 50.0 | 6.17 |
| K12/ENT-K12 | probiotic | | |
| | microorganism | | |
| Vitamin D3 premix | Active ingredient | 2.0 | 0.24 |
| Isomaltulose | Bulking agent | 734.16 | 90.64 |
| Pregelatinized corn starch | Binder | 7.42 | 0.92 |
| CompactCel ® LUB | Anticaking agent, | 8.1 | 0.99 |
| | lubricant | | |
| Strawberry flavor | Flavor | 8.0 | 0.99 |
| Stevia | Natural sweetener | 0.32 | 0.04 |
| Total | | 810 | 100 |

Of course, magnesium stearate can be substituted with a natural lubricant/anti-caking agent like a rice extract blend, for example the product Nu-MAG®, or the product CompactCel® LUB (CompactCel® F clear 290.02 LUB, Biogrund GmbH, Huenstetten, Germany), an oat fiber blend, for example the product CompactCel® LUB (CompactCel® F 200.28 LUB, Biogrund GmbH, Huenstetten, Germany) or further products, like potato starch, or gum Arabica, powdered cellulose, like JELUCEL®, native potato starch, inulin, rice fibers, or rice hulls, for example Nu-FLOW®, or CompactCel®FLO, but preferably with a rice extract blend, preferably with CompactCel® LUB, and/or HMPC can be substituted with a natural binder like CompactCel®DIS, NuBind®, or preferably pregelatinized corn starch. Furthermore, CompactCel® LUB and/or pregelatinized corn starch can be substituted with other natural lubricants/binders, like those mentioned above.

The compressed product of the present invention may also comprise silicon dioxide, but this can also be substituted with a natural anti-caking agent like rice hulls, for example CompactCel®FLO, or Nu-FLOW®. Preferably, the compressed product does not comprise magnesium stearate and silicon dioxide, but instead a rice extract blend.

In case more of the probiotic microorganisms are comprised in the compressed product, the amount of isomaltulose will be reduced so that the compressed product still weights about 810 mg. In case less of the probiotic microorganisms are comprised in the compressed product, the amount of isomaltulose will be increased so that the compressed product still weights about 810 mg. In case the compressed product of the present invention comprises more or less vitamin D3, or any other of the vitamins and/or minerals mentioned above, or in case vitamin D3 (or any other of the above-mentioned vitamins and/or minerals) is completely omitted, the amount of isomaltulose will be adapted accordingly.

In a most preferred embodiment, the compressed product of the present invention has the ingredients as listed in Table 2C.

As regards the further excipients and active ingredient, the powder product of the present invention comprises in a preferred embodiment 1% (15 mg) silicon dioxide, tricalcium phosphate, and Nu-Flow®, respectively, 0.67% (10 mg) aromatizing agent, preferably strawberry flavor, and 0.2% of a vitamin and/or mineral, preferably 5-10 μg, more preferably 4-6 μg vitamin D3 (cholecalciferol), which can be provided for example in a form of a premix (0.2%) which comprises 2.5 μg/mg vitamin D3. In one embodiment, the powder product further comprises FOS (32%, 480 mg).

An exemplarily powder product comprises the ingredients as shown in Table 2D.

TABLE 2D
Ingredients of an exemplarity isomaltulose/probiotics powder.

| | | Amount | Percentage |
|---|---|---|---|
| | Function | (mg) | (%) |
| Streptococcus salivarius | Active ingredient/ | 50.0 | 3.33 |
| K12/ENT-K12 | probiotic | | |
| | microorganism | | |
| FOS | Active ingredient | 480 | 32 |
| Isomaltulose | Bulking agent | 945 | 63 |
| Silicon dioxide | Anticaking agent | 15 | 1.0 |
| Strawberry flavor | Flavor | 10 | 0.67 |
| Total | | 1500 | 100 |

Of course, silicon dioxide can be substituted with tricalcium phosphate or a natural anti-caking agent, like rice hulls (Nu-Flow®), or CompactCel® FLO as explained above, and can also be completely omitted.

In case more of the probiotic microorganisms are comprised in the powder product, the amount of isomaltulose will be reduced so that the product still weights about 1500 mg. In case less of the probiotic microorganisms are comprised in the powder product, the amount of isomaltulose will be increased so that the product still weights about 1500 mg. In case the powder product of the present invention further comprises any of the vitamins and/or minerals mentioned above, preferably vitamin D3 (preferably in a final concentration of 4 to 6 μg), the amount of isomaltulose will be adapted accordingly.

The present invention further relates to a package comprising the product of the present invention. Preferably, the present invention further relates to a package comprising the compressed product of the present invention, for example a container, i.e. bottle or a blister pack.

Powder product are presented as single-dose or multidose preparations in suitable containers. Multidose oral powders are packed into a suitable bulk container, such as a wide-mouthed glass jar. They require the provision of a measuring device capable of delivering the quantity prescribed. Because of the difficulty in precisely measuring single doses from this type of preparation the constituents are usually relatively non-toxic medicaments with a large dose. Each dose of a single-dose powder is enclosed in an individual container, for example a sachet or a vial. Preferably, the powder product of the present invention is enclosed in a sachet. Accordingly, the present invention also relates to a package comprising the powder product of the present invention, for example a container, i.e. bottle or glass jar, a sachet or vial, preferably a sachet.

As mentioned above, probiotics are defined as live microorganisms that, when administered in adequate amounts, confer a health benefit on the host. The beneficial effects of probiotics may be mediated by a direct antagonistic effect against specific groups of undesired organisms, resulting in a decrease of their numbers, by an effect on the metabolism of such groups of organisms or by a general stimulatory effect on the immune system of animal or human hosts. Thus, the present invention relates to the use of the product for reducing the occurrence of infections with pathogens for example in the oral cavity, the respiratory tract, the gastrointestinal tract and the vagina or of the skin. Accordingly, the product of the present invention is used for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form in a subject.

In the present context, the expression “reducing the occurrence of infections” indicates that the above-mentioned infections or symptoms caused by the presence of pathogens occurs at a reduced frequency or seriousness as compared to a human or animal subject who or which is not being treated with the compressed product of the present invention. In the present context treatment is also to be construed as encompassing prevention or prophylaxis in addition to cure.

The invention also relates to a method for reducing the occurrence of infections with pathogens for example in the oral cavity, the respiratory tract, the gastrointestinal tract and the vagina or of the skin, wherein the method comprises administering the product according to the invention to a subject, preferably in the form of a compressed product, wherein the compressed product is a tablet, preferably a chewable tablet or lozenge and is administered orally, or in the form of a powder product, wherein the powder product is comprised in a sachet and is administered orally. Accordingly, the invention relates to a method for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form by administering the product according to the invention to a subject.

It is furthermore documented that probiotic microorganisms produce essential vitamins and nutrients required by the intestinal cells and furthermore assist with degradation of certain nutrients and even activate cell-mediated immune effector functions. Thus probiotic microorganisms can improve the general health status of a mammal.

A number of reports indicate that intake of probiotic microorganisms may not only reduce the occurrence of infections and are health promoting, but indeed contribute to the treatment of disease; see for example Wescombe et al., Future Microbiol. (2012) 7(12), 1355-1371; Zupancic et al., Probiotics Antimicrob Proteins (2017) 9(2), 102-110; Wilcox et al., Clin Microbiol Infect (2019) 25(6), 673-680; Marom et al., Medicine (Baltimore) (2016) 95(6), e2695; Clark, Curr Opin Immunol (2020) 66, 42-49; Bertuccioli et al., Nutrafoods (2019) 2, 80-88. Thus, the present invention relates to a product for use in the treatment of various diseases or conditions like otitis, preferably otitis media; upper respiratory tract infections, preferably tonsillitis or pharyngitis; lower respiratory tract infections, preferably bronchitis or pneumonia; diseases and inflammations of the oral cavity, preferably caries, oral mucositis, periodontitis, candidiasis, and/or oral lichen planus; halitosis; skin disorders, preferably acne and/or dermatitis; gastro-intestinal problems like diarrhea, gastroenteritis, irritable bowel syndrome (IBS), inflammatory bowel disease (IBD), urogenital infection; allergies, lactose intolerance or immune diseases preferably allergic rhinitis, or mastitis in a subject.

These health benefitting effects have been verified is various studies and described for example in Fijan (2014), Int J Environ Res Public Health. 11(5): 4745-4767; EP 1 483 366 B1; EP 2 581 461 B1; U.S. Pat. No. 6,994,848 B2; WO 2005/007178 A1; Di Pierro et al., Drug Healthc Patient Saf. 6 (2014), 15-20.

The invention also relates to a method for treatment of the above mentioned conditions/diseases in a subject, wherein the method comprises administering the product according to the invention to the subject.

The term “individual” or “subject” as used herein includes humans, horses, dogs, cats, pigs, sheep, cattle, goats but is not limited thereto. Preferably, the individual is a human. The product of the present invention can be administered to the individual at any age, e.g. childhood, adolescence, or adulthood.

In general, the amount of probiotics administered via the product of the present invention to the individual will be an amount of an active agent high enough to deliver the desired benefit, but low enough to avoid serious side effects. Specific dosages can vary widely according to various individual variables including size, weight, age, disease severity and responsiveness to therapy. Methods for determining the appropriate dosage may be determined by the consumer as they deem appropriate, or on a case-by-case basis by an attending a pharmacist or clinician. Such determinations are routine to one of ordinary skill in the art (see for example, Remington's Pharmaceutical Sciences, 8th ed., Gennaro, ed., Mack Publishing Company, Easton, Pa., 1990).

The product of the present invention may need to be administered to the patient once only or more usually repeatedly. Repeat prophylactic or therapeutic treatments may be once a month, once a week, once a day, twice a day, three times a day, four times a day, five times a day, six times a day or as may otherwise be required.

The present invention also relates to a method of producing a product which comprises at least probiotic bacteria as active ingredient and isomaltulose as excipient. The ingredients and features of the product to be produced with the method of the present invention are those as defined hereinbefore.

Methods of producing a powder product and of pressing tablets are known to the persons skilled in the art and thus, only the most important steps are described in the following.

The probiotic bacteria which are used in the method of the present invention can be provided in several forms, but preferably they are provided freeze-dried.

The method of the present invention comprises at least mixing the probiotic microorganisms with isomaltulose and filling the mixture into sachets (powder product of the present invention) or compressing the mixture to a compressed product. In one embodiment, the method of the present invention further comprises blending/homogenizing the mixture before filling the mixture into sachets or before compressing the mixture to the compressed product. This is performed to achieve blend uniformity and to uniformly distribute all ingredients. The optimum mixing time and speed to achieve a homogenous mixture can be evaluated by the person skilled in the art. For mixing and blending a fluidized bed granulator can be, and is preferably used.

In one embodiment, the method of the present invention further comprises analyzing the product, e.g. analyzing one, two, three, four, five, six, or all seven of the following parameters: CFU/g amount, water activity, disintegration time, average mass, resistance to crushing, appearance, and friability of the product.

In a preferred embodiment, the method for producing a compressed product of the present invention comprises the mixing of the probiotic bacteria with isomaltulose and the one or more further excipients as defined hereinbefore, in particular with a lubricant as defined above or with a lubricant and a binder as defined above, preferably wherein the lubricant is magnesium stearate, more preferably a natural substitute like a rice extract blend as defined above, and the binder is HMPC, more preferably a natural substitute like pregelatinized starch.

In another preferred embodiment, the method for producing a powder product of the present invention comprises the mixing of the probiotic bacteria with isomaltulose and with an anti-caking agent as defined above, preferably silicon dioxide and preferably also with FOS.

The lubricant magnesium stearate can also be substituted with a natural lubricant as defined hereinbefore and the anti-caking agent can be substituted with a natural anti-caking agent or ticalcium phosphate as defined hereinbefore.

In one embodiment, the method of the present invention further comprises the mixing of the above-mentioned ingredients with an aromatizing agent as mentioned before, like strawberry flavor, and optionally with a natural sweetener, preferably Stevia. Optionally, a further active ingredient, preferably a vitamin and/or mineral salt, most preferably vitamin D3 is also added and mixed with the probiotics (for example in form of a premix), the isomaltulose (and optionally FOS in case of the powder product) and the further excipient(s). Preferably, segment mixing is performed wherein the probiotic microorganisms and optionally the further active ingredient(s) are mixed stepwise with parts of the excipients.

The mixed and preferably blended powder, i.e. the powder product of the present invention can now be packed in suitable containers/bottles and/or filled into vials or sachets, preferably in sachets. In this case, the blended powder is analyzed preferably regarding its appearance, its CFU account and/or its water activity. This can be performed by methods known in the art and in particular by the methods as described in the Examples section.

Otherwise, the mixed and preferably blended powder is further used for manufacturing the compressed product of the present invention, i.e. the blended/homogenized powder is pressed. The compression force is a critical parameter since many probiotically active organisms, including the most interesting probiotic lactic bacteria, are highly sensitive to the pressure caused when formed into tablets by direct compression. Thus, a compression force has to be chosen which allows survival of the probiotics but which leads to a tablet with the desired properties, i.e. the properties as described above regarding water activity, disintegration time, average mass, resistance to crushing, CFU count and/or friability.

As a general rule: the lower the compression force the higher the survival rate. However, a low compression force results in less coherent tablets, but the use of isomaltulose results in a tablet with a good coherency and a high survival rate of the probiotic organisms as for example compared to tablets in which isomalt has been used; see the Examples. The compression force can be chosen between 1 to 50 kN/compressed product or kN/cm², respectively, preferably between 1 to 40 kN/compressed product or kN/cm², respectively, preferably between 1 to 30 kN/compressed product or kN/cm², respectively, preferably between 1 to 20 kN/compressed product or kN/cm², respectively, more preferably between 1 to 10 kN/compressed product or kN/cm², respectively, more preferably between to 6 to 10 kN/compressed product or kN/cm², respectively and most preferably around 7 to 8 kN/compressed product or kN/cm², respectively.

Also the dwell-time, i.e. the time during which the compression is maximal, needs to be considered. According to a preferred embodiment the compression time, i.e. the short period of time wherein the compressible formulation is compressed in the tablet press, is less than 1 s, preferably the time is less than 0.5 s, more preferably less than 0.1 s and most preferably less than 0.08 s.

Another point is the heat development during compression. Since probiotics are heat sensitive, the generation of excessive heat during compression should be avoided. Thus, the tablet press is run at a low speed in order to avoid strong heat development. The preferred speed can be tested by a person skilled in the art but preferably the tablet press is run at such speed that about 600 tablets are pressed per minute.

After compression, the compressed product is preferably analyzed regarding its CFU count, disintegration time, its average mass, its resistance to crushing, its friability, its appearance, and/or its water activity. This can be performed by methods known in the art and in particular by the methods as described in the Examples section.

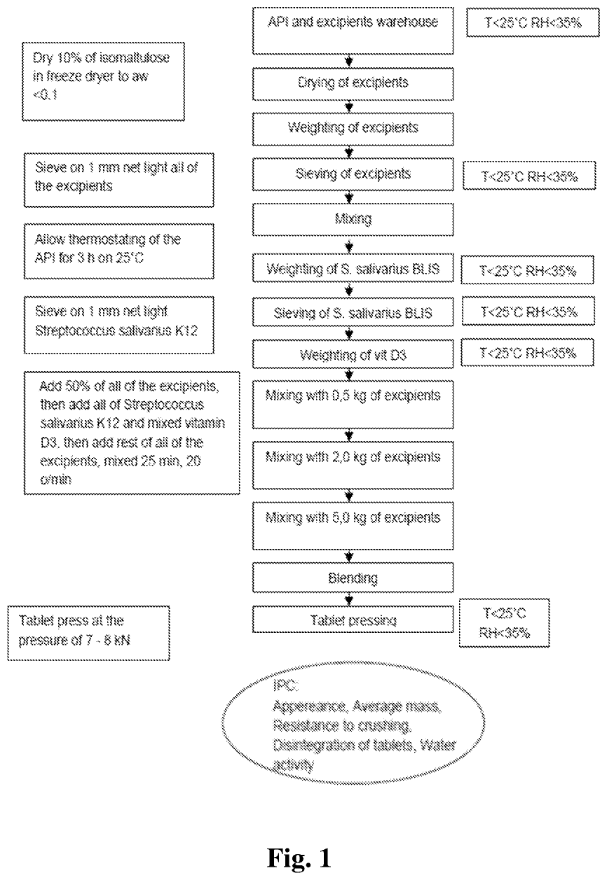

In one embodiment, the method of the present invention does not comprise a granulation step, i.e. no slugging and/or roller compaction/ribbon blending is performed, but the blended mixture is directly compressed into the final compressed product. Accordingly, the method of the present invention comprises in one embodiment at least the following steps: (i) mixing the probiotic microorganisms with isomaltulose and with one or more further excipients as defined above, preferably with a lubricant as defined above, preferably with magnesium stearate, and optionally with one or more further active ingredients as defined above, preferably with vitamin D3; (ii) blending the mixture; and (iii) compressing the mixture to a compressed product; see also FIG. 1.

Alternatively, the method of producing the compressed product comprises a briquetting step as described in EP 22 168 218.0, wherein the isomaltulose and preferably the other excipients mentioned above are first compressed to briquettes, wherein afterward the briquettes are fractionized and grinded, followed by the addition of the probiotic microorganisms to the grinded briquettes, mixing and preferably blending/homogenizing. Accordingly, the method of the present invention comprises in one embodiment at least the following steps: (i) mixing the isomaltulose with one or more further excipients as defined above, preferably with a lubricant as defined above, preferably with magnesium stearate, and most preferably with a natural substitute, like a rice extract blend as defined above; (ii) compression of the mixed excipients to briquetts; (iii) fractionizing and grinding the briquetts; (iv) addition of the probiotic microorganisms to the grinded briquettes and optionally addition of one or more further active ingredients as defined above, preferably vitamin D3; (v) homogenizing/blending the mixture; and (vi) compressing the mixture to a compressed product. Those steps are in detail described in EP 22 168 218.0, which content is herein incorporated by reference.

Alternatively, the method of producing the compressed product comprises a wet granulation step. Wet granulation can in general be performed with water or ethanol as a solvent and using various binders (HPMC, PVP K-25, PVP K-30, PEG 4000, PEG 6000, microcrystalline cellulose, starch, and its derivatives). The wet granulation process is performed in a granulation device by spraying a binder solution on excipients, under controlled pressure conditions (0.15-0.18 MPa) under which the liquid is sprayed. Granulation devices can be for example a high-shear granulator, a twin screw granulator or a fluidized bed granulator). During this procedure, wet granules are formed. After the completion of the spraying procedure, the granules are dried at a controlled temperature of 40±2° C., for about 30 minutes. The resulting granulate is cooled to room temperature and water activity and relative humidity are less than 0.25 and less than 1.5%, respectively. With the prepared granulate, the active ingredients are homogenized and tableting is started.

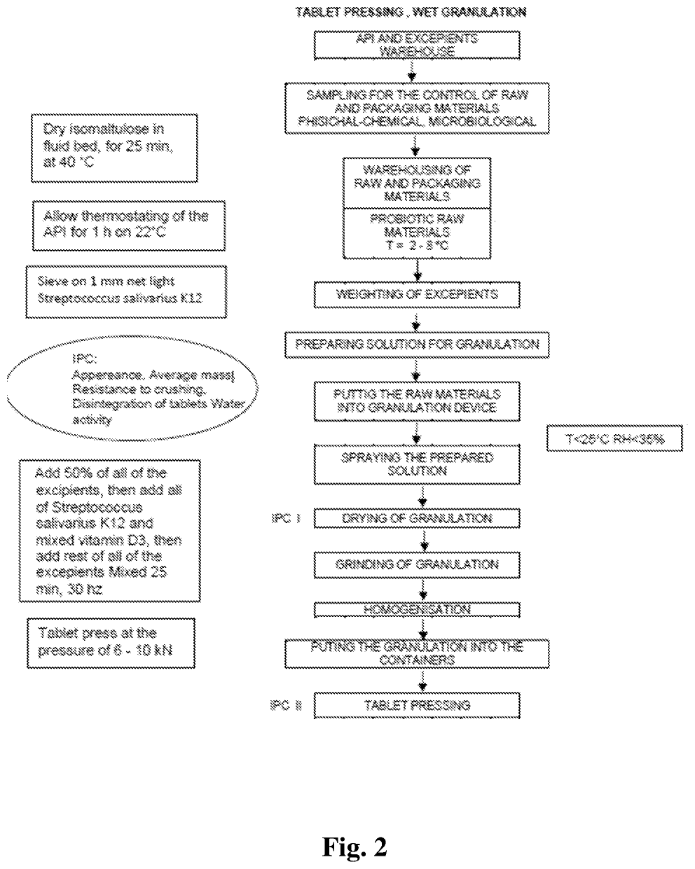

Accordingly, the method of the present invention comprises in one embodiment at least the following steps: (i) mixing the isomaltulose with one or more further excipients as defined above, preferably with a lubricant as defined above, preferably with magnesium stearate, more preferably a natural substitute, preferably a rice extract blend as defined above; (ii) spraying a binder solution on the excipients, preferably wherein the binder solution is composed of water and a binder as defined above, preferably wherein the binder is HMPC or pregelatinized corn starch, most preferably pregelatinized corn starch, and preferably wherein the spraying is performed under controlled pressure conditions (0.15-0.18 MPa), thereby forming the wet granules; (iii) drying the granules, preferably at a controlled temperature of 40±2° C., for about 30 minutes, preferably until a water activity and relative humidity of less than 0.25 and less than 1.5%, respectively, is reached; (iv) grinding/milling the granules; (v) addition of the probiotic microorganisms to the grinded granules and optionally addition of one or more further active ingredients as defined above (preferably vitamin D3); (vi) homogenizing/blending the mixture; and (vii) compressing the mixture to a compressed product; see also FIG. 2.

The excipients which are used for the preparation of the product of the present invention are preferably dried before further processing. In a preferred embodiment, only 1-10% of the isomaltulose are dried in a freeze dryer to a water activity of about ≤0.1% and the other 90-99% are not dried; more preferably, only 10% of the isomaltulose are dried in a freeze dryer to a water activity of about ≤0.1% and the other 90% are not dried (dry compression). Alternatively all of the isomaltulose is dried in a fluid bed for about 25 minutes at 40° C. (wet granulation).

As mentioned with regard to the product of the present invention above, the mixture used in the method of the present invention for preparing said product comprises at least 10% and up to 97% isomaltulose. Preferably, the amount of isomaltulose is between 50% and 97%, more preferably between 58% and 96%. As regards the compressed product, the amount of isomaltulose in the mixture is preferably between 80% and 97%, more preferably between 82% and 92%, more preferably 82.8%, 83.2%, 85.3%, 85.7%, 90.2%, 90.6%, 91.4%, 91.5%, or 91.9%. As regards the powder product, the amount of isomaltulose in the mixture is preferably between 50% and 97%, more preferably between 58% and 96%. In particular, the amount of isomaltulose is between 58% and 64%, and most preferably 58%, 60%, 62%, 63% or 64% (in case FOS is additionally present in the powder product), or between 92% and 96% and most preferably 90%, 92%, 94%, 95%, or 96% (in case no FOS is present in the powder product). The probiotic microorganisms will comprise at least 0.3% and up to 20% of the mixture. As regards the compressed product, the probiotics will comprise 0.6% to 20% of the mixture and preferably, the content of the probiotic microorganisms is between 3% and 15%, more preferably about 6.2%, 7.4%, 12.3% or 14.8%. As regards the powder product, the probiotic will comprise at least 0.3% and up to 10% of the mixture and preferably, the content of the probiotic microorganisms is between 2% and 8%, more preferably about 3% (2.7 or 3.3%), 4%, 7% (6.6%), or 8%.

As regards the further excipients and active ingredient (next to isomaltulose and the probiotic microorganisms), the mixture used in the method of the present invention to produce a compressed product, in particular by dry compression, comprises in a preferred embodiment 1% of a lubricant/anti-caking agent, preferably magnesium stearate and more preferably its natural substitute, respectively, as defined above, preferably a rice extract blend as defined above like Nu-MAG® or CompactCel®LUB, preferably CompactCel®LUB, and 1% aromatizing agent, preferably strawberry flavor, and optionally 0.2% or preferably 0.4% of vitamin D3 premix (cholecalciferol), which comprises 2.5 μg/mg vitamin D3, and optionally, but preferably 0.04% of a natural sweetener, preferably Stevia.

As regards the further excipients and active ingredient (next to isomaltulose and the probiotic microorganisms), the mixture used in the method of the present invention to produce a compressed product, in particular by compression including a wet granulation step, comprises in a preferred embodiment 1% of a lubricant/anti-caking agent, preferably magnesium stearate and more preferably its natural substitute, respectively, as defined above, preferably a rice extract blend as defined above like Nu-MAG® or CompactCel®LUB, preferably CompactCel®LUB, 1% aromatizing agent, preferably strawberry flavor, 0.1% to 0.9% of a binder, preferably 0.1% HMPC or a natural substitute as defined above, preferably CompactCel®DIS, NuBind® or pregelatinized starch, preferably 0.9% pregelatinized corn starch, and optionally 0.2% or preferably 0.4% of vitamin D3 premix (cholecalciferol), which comprises 2.5 μg/mg vitamin D3, and optionally, but preferably 0.04% of a natural sweetener, preferably Stevia.

As regards the further excipients and active ingredient (next to isomaltulose and the probiotic microorganisms), the powder product of the present invention comprises in a preferred embodiment 1% of an anti-caking agent, preferably silicon dioxide, tricalcium phosphate, and a natural substitute, respectively, as defined above, preferably Nu-Flow® or CompactCel®FLO, 0.67% aromatizing agent, preferably strawberry flavor, and optionally 0.4% or preferably 0.2% of vitamin D3 premix (cholecalciferol), which comprises 2.5 μg/mg vitamin D3. In one embodiment, the powder product further comprises FOS (32%), and optionally 0.04% of a natural sweetener, preferably Stevia.

Before the mixing and blending steps with the active ingredient(s) are performed, the excipients are weighted, sieved, preferably through a 1 mm net and mixed. Furthermore, the probiotic microorganisms are weighted and sieved, preferably through a 1 mm net and optionally are thermostabilized for 1 to 3 hours before the weighting and sieving step. In case a product in produced which comprises further active ingredients, in particular which comprises vitamin D3, the vitamin D3 premix is also weighted before mixing.

In order to achieve a low water activity of the product which is important for its shelf-life, most of the steps are performed at room temperature and at a relative humidity of about ≤35% as can be derived from FIG. 1 and FIG. 2. Room temperature can be defined as a temperature of ≤25° C. and more preferably of 15° C. to 25° C.

In a preferred embodiment, the product to be produced with the method of the present invention is the product of the present invention. Furthermore, the present invention relates to the product as obtained with the method of the present invention, preferably which has the characteristics as mentioned above, i.e. the water activity, disintegration time, average mass, resistance to crushing, CFU count and/or friability as defined above and shown in the Examples.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific Examples which are provided herein for purposes of illustration only and are not intended to limit the scope of the invention.

### EXAMPLES

Material and Methods

Preparation of the Compressed Product The preparation of the compressed product is performed according to the schema depicted in FIG. 1 (dry compression) or FIG. 2 (wet granulation) and is exemplarily described for tablets comprising Streptococcus salivarius K12, isomaltulose, magnesium stearate, strawberry flavor, and vitamin D3 or for tablets comprising Streptococcus salivarius K12, isomaltulose, magnesium stearate, HMPC, strawberry flavor, and vitamin D3. However, the same process is used for tablets of the present invention which are defined hereinbefore.

As regards the dry compression method, the excipients (here: isomaltulose, magnesium stearate, strawberry flavor) are dried, weighted, sieved through a 1 mm net and mixed. In particular, 10% of the isomaltulose are dried in a freeze-dryer to a water activity of <0.1, wherein 90% of the isomaltulose are not dried, and the sieving is performed at room temperature, in particular at <25° C., and at a relative humidity of <35%.

S. salivarius K12 is used as freeze-dried product and before further processing, it allowed to thermostate for 3 h at 25° C. Afterwards, S. salivarius K12 is weighted and sieved through a 1 mm net, and vitamin D3 is weighted, wherein those steps are performed at room temperature, in particular at <25° C., and at a relative humidity of <35%.

In a next step, 10% of the excipients are mixed with S. salivarius K12 and vitamin D3. Afterwards, further 40% of the excipients are added and mixed and finally the remaining 50% of the excipients are added and mixed. Mixing is performed for about 25 minutes at 20 o/min. The next step comprises blending and tablet pressing. Tablet pressing is performed at about 7 to 8 kN and the tablet press is run at low speed in order to avoid strong heat production, for example at a speed to produce 600 tablets/min. The tablet pressing step is also performed at room temperature, in particular at <25° C., and at a relative humidity of <35%. The pressed tablets are analyzed with regard to their appearance, average mass, resistance to crushing, disintegration time, friability and/or water activity.

The concentration of the excipients and the active pharmaceutical ingredients in this exemplarily process are as follows:
6.2% Streptococcus salivarius K12,
91.5% isomaltulose,
1% magnesium stearate,
1% strawberry flavor, and
0.4% vitamin D3 premix.

The vitamin D3 premix can be substituted with isomaltulose and if different amounts of the probiotic microorganism are used, the concentration of isomaltulose is adapted accordingly.

As regards the wet granulation compression method, at least the isomaltulose is dried. In particular, the isomaltulose is dried for 25 min at 40° C. in a fluid bed. Afterwards, the excipients (here: isomaltulose, magnesium stearate, HMPC, strawberry flavor) are weighted.

S. salivarius K12 is used as freeze-dried product and before further processing, it allowed to thermostate for 1 h at 22° C. Afterwards, S. salivarius K12 is weighted and sieved through a 1 mm net, and vitamin D3 is weighted.

In a next step, a solution for granulation is prepared, i.e. water is mixed with the binder HPMC. Afterwards, the isomaltulose and magnesium stearate are put into the granulation device, i.e. isomaltulose and magnesium stearate are mixed. This is followed by spraying the binder solution on the excipients under controlled pressure conditions (0.15-0.18 MPa), thereby forming the wet granules. Afterwards, the granulate is dried at a controlled temperature of 40±20° C., for about 30 minutes until a water activity and relative humidity of less than 0.25 and less than 1.5%, respectively, is reached. This is followed by cooling and grinding the granulate and by addition of S. salivarius K12 and vitamin to the grinded granulate. In the next step, 50% of the excipients are mixed with S. salivarius K12 and vitamin D3. Afterwards, the remaining excipients are added and mixed. Mixing is performed for about 25 minutes at 30 hz. The next step comprises tablet pressing. Tablet pressing is performed at about 6 to 10 kN and the tablet press is run at low speed in order to avoid strong heat production, for example at a speed to produce 600 tablets/min. The tablet pressing step is also performed at room temperature, in particular at <25° C., and at a relative humidity of <35%. The pressed tablets are analyzed with regard to their appearance, average mass, resistance to crushing, disintegration time, friability and/or water activity.

The concentration of the excipients and the active pharmaceutical ingredients in this exemplarily process are as follows:
6.2% Streptococcus salivarius K12,
91.4% isomaltulose,
0.1% HMPC
1% magnesium stearate,
1% strawberry flavor, and
0.4% vitamin D3 premix.

The vitamin D3 premix can be substituted with isomaltulose and if different amounts of the probiotic microorganism are used, the concentration of isomaltulose is adapted accordingly.

Preparation of the Powder Product

The preparation of the powder product having a composition as described hereinbefore comprises in principle the same steps than the method for the preparation of the compressed product by dry compression explained above until and including the blending step. The powder product is analyzed with regard to its appearance, average mass, and/or water activity.

Determination of Colony Forming Units (CFU)

Determination of CFUs of S. salivarius, L. casei and L. rhamnosis is performed as described in “Istituto Superiore di Sanità, Metodi microbiologici tradizionali e metodi molecolari per l'analisi degli integratori alimentari a base di o con, probiotici per uso umano, by Paolo Aureli, Alfonsina Fiore, Concetta Scalfaro, Giovanna Franciosa, 2008, ii, 63 p. Rapporti ISTISAN 08/36, ISSN 1123-3117”, in particular in chapters 21 and 24.

Determination of the Hardness of the Compressed Product

The determination of the hardness of the compressed product, i.e. its resistance to crushing, is performed as described in chapter 2.9.8 of the European Pharmacopoeia 6.0 by Council of Europe; 6th Edition; published on May 10, 2008; ISBN-10: 9287160546; ISBN-13: 978-9287160546.

Determination of the Disintegration Time of the Compressed Product

The determination of the disintegration time of the compressed product is performed as described in chapter 2.9.1 of the European Pharmacopoeia 6.0 by Council of Europe; 6th Edition; published on May 10, 2008; ISBN-10: 9287160546; ISBN-13: 978-9287160546.

Determination of the Water Activity

The determination of the water activity is performed using the HygroLab 3 of Rotronic AG Bassersdorf, Switzerland, and measurement is performed according to the user manual.

Determination of the Friability of the Compressed Product

The determination of the friability of the compressed product is performed as described in chapter 2.9.7 of the European Pharmacopoeia 6.0 by Council of Europe; 6th Edition; published on May 10, 2008; ISBN-10: 9287160546; ISBN-13: 978-9287160546.

### Example 1: Comparison of a Lozenge Comprising S. salivarius K12 and Isomaltulose with a Lozenge Comprising S. salivarius K12 and Isomalt

In a first approach, isomaltulose lozenges comprising about 6.2% Streptococcus salivarius K12, 91.5% isomaltulose, 1% magnesium stearate, 1% strawberry flavor, and 0.4% vitamin D3 premix have been compared to corresponding isomalt lozenges, which comprise isomalt as bulking agent instead of isomaltulose. In particular, the lozenges comprise 50 mg S. salivarius K12 and have been analyzed regarding their hardness, disintegration time, friability, water activity and CFUs of S. salivarius K12. The results are shown in Tables 3 and 4.

As can be derived from Tables 3 and 4, the isomaltulose lozenges are not as hard as the isomalt lozenges. However, remarkably the CFUs are in average much higher in the isomaltulose lozenges than in the isomalt lozenges. In particular, although the same concentration of freeze-dried S. salivarius K12 has been used as raw material, more viable bacteria are present in the isomaltulose lozenges in comparison to the corresponding lozenges with isomalt. Furthermore, the disintegration time of the isomaltulose lozenges is higher than the disintegration time of the isomalt lozenges. The friability of both lozenges is comparable, but the water activity of the isomalt lozenges is slightly higher.

TABLE 3
Characteristics of isomaltulose lozenges comprising 50 mg S. salivarius K12. Two
different batches are exemplarily shown (first batch in bold, second batch in italics, wherein
values in bold and italics indicate that the values of the first and the second batch are the same).

| Time | Average | | Disintegration | Friability | Water | Premix | CFU/g |
|---|---|---|---|---|---|---|---|
| [months] | mass [mg] | Hardness [Newton] | time [min] | [%] | activity [Aw] | CFU/g K12 | K12 |
| 0 | 807/946 | [symbol] | > [symbol] | 1.23/1.5 | 0.107/0.136 | 1.30E+10/ | 1.22E+10/ |
| | | | | | | 1.04E+10 | 9.60E+9 |
| 3 | | | | | 0.166/0.174 | | 8.00E+09/ |
| | | | | | | | 7.60E+9 |
| 6 | | | | | 0.178/0.192 | | 7.03E+09/ |
| | | | | | | | 6.90E+9 |
| 9 | | | | | 0.184/0.22 | | [symbol] |
| 12 | | | | | 0.182/0.217 | | 3.04E+09/ |
| | | | | | | | 4.50E+9 |

TABLE 4
Characteristics of isomalt lozenges comprising 50 mg S. salivarius K12. Two different
batches are exemplarily shown (first batch in bold, second batch in italics, wherein values in
bold and italics indicate that the values of the first and the second batch are the same).

| Time | Average | Hardness | Disintegration | Friability | Water | Premix | |
|---|---|---|---|---|---|---|---|
| [months] | mass [mg] | [Newton] | time [min] | [%] | activity [Aw] | CFU/g K12 | CFU/g K12 |
| 0 | 800/803 | [symbol] | [symbol] | 1.29/ | 0.159/0.175 | 7.62E+09/ | 7.08E+09/ |
| | | | | 1.38 | | 6.52E+09 | 6.02E+09 |
| 3 | | | | | 0.151/0.219 | | 6.58E+09/ |
| | | | | | | | 6.02E+09 |
| 6 | | | | | 0.185/0.213 | | 6.07E+09/ |
| | | | | | | | 5.10E+09 |
| 9 | | | | | 0.19/0.205 | | [symbol] |
| 12 | | | | | 0.171/0.206 | | 5.18E+09/ |
| | | | | | | | 3.53E+09 |

### Example 2: Comparison of a Lozenge Comprising S. salivarius K12, a Lactobacillus Strain and Isomaltulose with a Lozenge Comprising S. salivarius K12, a Lactobacillus Strain and Isomalt

In a second approach, isomaltulose lozenges comprising S. salivarius K12 and L. rhamnosus LGG or S. salivarius K12 and L. casei in different concentrations (5%, 6.2%, 7.4%, 9.3%) isomaltulose (92.7%, 91.5%, 88.4%, 85.3%) magnesium stearate (1%), strawberry flavor (1%), and vitamin D3 premix (0.4%) have been compared to corresponding isomalt lozenges, which comprise isomalt as bulking agent instead of isomaltulose. In particular, the lozenges have been analyzed regarding their hardness, disintegration time, friability, water activity and CFUs of S. salivarius K12 directly after compressing (time point 0). The results are shown in Tables 5 and 6.

TABLE 5
Characteristics of isomaltulose lozenges comprising different amounts of S. salivarius
K12, L. rhamnosus LGG, and L. casei 431 measured directly after compression (time point 0).

| | | | | | Water | | | |
|---|---|---|---|---|---|---|---|---|
| | Average | Hardness | Disint. | Friability | activity | CFU/g | CFU/g | CFU/g |
| Dose | mass [mg] | [Newton] | time [min] | [%] | [Aw] | K12 | LGG | casei |
| K12-50 mg; | 803 | 83 | 11 | 1.25 | 0.129 | 7.03E+09 | 1.05E+10 | |
| LGG-50 mg | | | | | | | | |
| K12-25 mg; | 803 | 82 | 10 | 1.24 | 0.14 | 3.13E+09 | 1.33E+10 | |
| LGG-50 mg | | | | | | | | |
| K12-30 mg; | 803 | 83 | 10 | 1.25 | 0.149 | 5.08E+09 | 7.09E+09 | |
| LGG-30 mg | | | | | | | | |
| K12-30 mg; | 803 | 82 | 11 | 1.26 | 0.137 | 4.95E+09 | | 3.25E+09 |
| casei-30 mg | | | | | | | | |
| K12-20 mg; | 803 | 85 | 10 | 1.24 | 0.144 | 3.05E+09 | 3.54E+09 | |
| LGG-20 mg | | | | | | | | |

TABLE 6
Characteristics of isomalt lozenges comprising different amounts of S. salivarius K12,
L. rhamnosus LGG, and L. casei 431 measured directly after compression (time point 0).

| | | | | | Water | | | |
|---|---|---|---|---|---|---|---|---|
| | Average | Hardness | Disint. | Friability | activity | CFU/g | CFU/g | CFU/g |
| Dose | mass [mg] | [Newton] | time [min] | [%] | [Aw] | K12 | LGG | casei |
| K12-50 mg; | 804 | 84 | 5 | 1.24 | 0.182 | 7.23E+09 | 8.15E+09 | |
| LGG-50 mg | | | | | | | | |
| K12-25 mg; | 803 | 84 | 5 | 1.24 | 0.193 | 3.74E+09 | 1.06E+10 | |
| LGG-50 mg | | | | | | | | |
| K12-30 mg; | 803 | 84 | 5 | 1.24 | 0.189 | 4.43E+09 | 7.12E+09 | |
| LGG-30 mg | | | | | | | | |
| K12-30 mg; | 803 | 85 | 5 | 1.24 | 0.191 | 5.10E+09 | | 3.34E+09 |
| L. casei-30 mg | | | | | | | | |
| K12-20 mg; | 803 | 85 | 5 | 1.25 | 0.195 | 3.78E+09 | 4.58E+09 | |
| LGG-20 mg | | | | | | | | |

As can be derived from Tables 5 and 6, the hardness, the CFU amount of the probiotics and the friability of both isomaltulose and isomalt lozenges is similar. The disintegration time of the isomaltulose lozenges is higher than the disintegration time of the isomalt lozenges and the water activity of the isomaltulose lozenges is lower.

### Example 3: Comparison of a Powder Product Comprising S. salivarius K12 and Isomaltulose with a Powder Product Comprising S. salivarius K12 and Maltodextrin

In a further approach, isomaltulose sachets comprising about 3.33% Streptococcus salivarius K12 (50 mg), 32% FOS (480 mg), 63% isomaltulose (945 mg), 1% silicon dioxide (15 mg), 0.67% strawberry flavor (10 mg), and about 4-6 μg vitamin D3 premix have been compared to corresponding maltodextrin lozenges, which comprise maltodextrin as bulking agent instead of isomaltulose. In particular, the sachets have been analyzed regarding their water activity and CFUs of S. salivarius K12 and the exemplarily results of each one batch are shown in Tables 7 and 8.

TABLE 7
Characteristics of isomaltulose sachets
comprising 50 mg S. salivarius K12.

| Time [months] | Water activity [Aw] | CFU/g K12 |
|---|---|---|
| 0 | 0.127 | 8.37E+09 |
| 6 | 0.13 | 7.07E+09 |

TABLE 8
Characteristics of maltodextrin sachets
comprising 50 mg S. salivarius K12.

| Time [months] | Water activity [Aw] | CFU/g K12 |
|---|---|---|
| 0 | 0.12 | 9.26E+09 |
| 6 | 0.117 | 8.34E+09 |
| 12 | 0.247 | 7.40E+09 |
| 18 | 0.238 | 4.35E+09 |
| 24 | 0.157 | 1.35E+09 |

As can be derived from Tables 7 and 8, both sachet formulations are storage stable since the water activity remains low and the CFU count high during storage. Thus, isomaltulose is as is suitable as excipient than the commonly used maltodextrin.

1. A product which comprises at least probiotic microorganisms as active ingredient and isomaltulose as excipient.

2. The product of claim 1 which is (i) a compressed product, preferably a tablet, more preferably a lozenge or a chewable tablet; or (ii) a powder product.

3. The product of claim 1, wherein the probiotic microorganisms are lactic acid bacteria, preferably of the genus Streptococcus, Lactobacillus, Lactococcus, Enterococcus, and/or Bifidobacterium, preferably wherein the probiotic microorganisms are selected from the group consisting of Streptococcus salivarius, preferably Streptococcus salivarius K12, Streptococcus salivarius ENT-K12, or Streptococcus salivarius M18; Lactobacillus rhamnosus, preferably Lactobacillus rhamnosus LGG; Lactobacillus casei; Lactobacillus paracasei; Lactobacillus fermentum; Lactobacillus crispatus; Lactobacillus plantarum; Bifidobacterium animalis subsp. lactis; Lactococcus lactis; Enterococcus faecalis; Lactobacillus reuteri; either alone or in combination.

4. The product of claim 1, which further comprises one or more excipients, preferably a lubricant and/or an anti-caking agent, and/or a binder, and/or an aromatizing agent, preferably wherein the lubricant is magnesium stearate or a natural substitute, wherein the anti-caking agent is silicon dioxide, tricalcium phosphate or a natural substitute, wherein the binder is HMPC or a natural substitute, and/or wherein the aromatizing agent is a flavor, preferably strawberry flavor, and optionally wherein the product further comprises a natural sweetener, and/or one or more active ingredients, preferably vitamins, minerals and/or fructooligosaccharides, preferably wherein the vitamin is vitamin D3.

5. The product of claim 1, wherein the product is
(i) a compressed product and further comprises a lubricant/an anti-caking agent, preferably wherein the lubricant/anti-caking agent is magnesium stearate or a natural substitute; an aromatizing agent, preferably wherein the aromatizing agent is a flavor, preferably strawberry flavor; and optionally a natural sweetener and/or one or more active ingredients, preferably a vitamin, preferably vitamin D3;
(ii) a compressed product and further comprises a lubricant/an anti-caking agent, preferably wherein the lubricant/anti-caking agent is magnesium stearate or a natural substitute; a binder, preferably wherein the binder is hydroxypropyl methylcellulose (HMPC) or a natural substitute; an aromatizing agent, preferably wherein the aromatizing agent is a flavor, preferably strawberry flavor; and optionally a natural sweetener, and/or one or more active ingredients, preferably a vitamin, preferably vitamin D3;
(iii) a powder product and further comprises an anti-caking agent, preferably wherein the anti-caking agent is silicon dioxide, tricalcium phosphate or a natural substitute; an aromatizing agent, preferably wherein the aromatizing agent is a flavor, preferably strawberry flavor; fructooligosaccharides; and optionally a natural sweetener and/or one or more active ingredients, preferably a vitamin, preferably vitamin D3.

6. The product of claim 1, wherein the product is a compressed product and further comprises a rice extract blend as lubricant/anti-caking agent, pregelatinized corn starch as binder, a flavor as aromatizing agent, preferably strawberry flavor, a natural sweetener, preferably Stevia, and vitamin D3 as further active ingredient.

7. The product of claim 1, wherein the product is
(i) a compressed product and the content of isomaltulose is at least 80%, preferably between 80% and 97%, more preferably between 82% and 92%, and/or wherein the content of the probiotic microorganisms is between 0.6% and 20%, preferably between 3% and 15%;
(ii) a powder product and the content of isomaltulose is at least 50%, preferably between 50% and 97%, more preferably between 58% and 96%, most preferably between 58% and 64% or between 92% and 96%, and/or wherein the content of the probiotic microorganism is between 0.3% and 11%, preferably between 2% and 8%; and/or
wherein the product comprises between 5 mg and 160 mg, preferably between 30 mg and 120 mg, more preferably 40 mg, 50 mg, 60 mg, 100 mg or 120 mg of the probiotic microorganisms.

8. The product of claim 1, which is a compressed product and which has a disintegration time of more than 5 minutes, preferably between 10 and 30 minutes, more preferably between 10 and 11 minutes and/or which has a friability of about 1.2% to 1.5%, preferably of about 1.25%.

9. A method of preparing a product comprising at least probiotic microorganisms as active ingredient, the method comprising utilizing isomaltulose as excipient.

10. A package comprising the product of claim 1.

11. A method of treating otitis, preferably otitis media; upper respiratory tract infections, preferably tonsillitis or pharyngitis; lower respiratory tract infections, preferably bronchitis or pneumonia; diseases and inflammations of the oral cavity, preferably caries, oral mucositis, periodontitis, candidiasis, and/or oral lichen planus; halitosis; skin disorders, preferably acne and/or dermatitis; gastro-intestinal problems; allergies or immune diseases preferably allergic rhinitis, or mastitis in a subject, the method comprising administering the product of claim 1 or a package comprising the product to the subject.

12. A method for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form in a subject, the method comprising administering the product of claim 1 or a package comprising the product to the subject.

13. A method of producing a product which comprises at least probiotic microorganisms as active ingredient and isomaltulose as excipient, wherein the method comprises at least the following steps:
(i) mixing the probiotic microorganisms with isomaltulose and with one or more further excipients and optionally with one or more further active ingredients; and
(ii) homogenizing the mixture; and
(ii) compressing the mixture to a compressed product, preferably wherein compression is performed with a pressure of 6 to 10 kN/compressed product, or filling the mixture into sachets, and optionally
(iii) analyzing the CFU/g amount and/or the water activity; and optionally the disintegration time, the average mass, the resistance to crushing, the appearance, the friability, and/or the water activity of the product.

14. The method of claim 13, wherein step (i) is composed of the following steps:
(a) mixing the isomaltulose with one or more further excipients, preferably with a lubricant/anti-caking agent, preferably with magnesium stearate;
(b) spraying a binder solution on the mixed excipients, preferably wherein the binder solution is composed of water and a binder, preferably wherein the binder is HMPC or pregelatinized starch, most preferably pregelatinized starch, thereby forming wet granules;
(c) drying the granules, preferably at a temperature of 40±2° C. for about 30 minutes;
(d) grinding the granules;
(e) adding the probiotic microorganisms to the grinded granules and optionally adding one or more further active ingredients.

15. The method of claim 13, wherein the probiotic microorganisms are lactic acid bacteria, preferably of the genus Streptococcus, Lactobacillus, Lactococcus, Enterococcus, and/or Bifidobacterium, preferably wherein the probiotic microorganisms are selected from the group consisting of Streptococcus salivarius, preferably Streptococcus salivarius K12, Streptococcus salivarius ENT-K12, or Streptococcus salivarius M18; Lactobacillus rhamnosus, preferably Lactobacillus rhamnosus LGG; Lactobacillus casei; Lactobacillus paracasei; Lactobacillus fermentum; Lactobacillus crispatus; Lactobacillus plantarum; Bifidobacterium animalis subsp. lactis; Lactococcus lactis; Enterococcus faecalis; Lactobacillus reuteri; either alone or in combination.

16. The A product obtainable by the method of claim 13.
